(19) United States
(12) Patent Application Publication
(10) Pub. No.: US 2021/0095310 A1
(43) Pub. Date: Apr. 1, 2021

(54) MICRORNA REGULATED EXPRESSION VECTORS, METHODS OF MAKING, AND USES THEREOF

(71) Applicant: Onconetics Pharmaceuticals, Inc., Mill Valley, CA (US)

(72) Inventors: Lukas DC GRUENERT, Mill Valley, CA (US); Gabriel HITCHCOCK, Mill Valley, CA (US); Roy Geoffrey SARGENT, San Lorenzo, CA (US)

(21) Appl. No.: 17/067,572

(22) Filed: Oct. 9, 2020

Related U.S. Application Data
(63) Continuation of application No. PCT/US2019/026790, filed on Apr. 10, 2019.
(60) Provisional application No. 62/655,619, filed on Apr. 10, 2018.

(51) Int. Cl. C12N 15/85; A61K 48/00; C12Q 1/6886; C12Q 1/6897; A61P 35/00

(52) U.S. Cl. CPC C12N 15/85; A61K 48/0058; C12Q 1/6886; C12N 2840/007; A61P 35/00; C12N 2810/10; C12Q 1/6897

(57) ABSTRACT

Provided herein are vectors, compositions and methods for treating and diagnosing breast cancer. In exemplary embodiments, the present disclosure provides a vector for the expression of a therapeutic protein, wherein the vector comprises a microRNA binding domain (MBD) that facilitates the expression of the therapeutic protein in breast cancer cells and inhibits the expression of the therapeutic protein in non-breast cancer cells. The present disclosure also provides compositions comprising the vectors and methods of using the vectors for treating and/or diagnosing breast cancer.

### CROSS-REFERENCE TO RELATED APPLICATIONS

The present application is a continuation of International Patent Application No. PCT/US2019/026790, filed Apr. 10, 2019, which claims the benefit of priority to U.S. Provisional Application No. 62/655,619, filed on Apr. 10, 2018, the contents of each of which are hereby incorporated by reference in their entireties.

### BACKGROUND OF THE DISCLOSURE

Current treatments for cancer have side effects that reduce the efficacy of treatments. One of the side effects is the toxicity of the treatment on healthy cells. Thus, there is an unmet need to provide therapeutic compositions and methods where the therapeutic agent is specifically expressed in target cancer cells but not in healthy cells.

### BRIEF SUMMARY OF THE DISCLOSURE

Provided herein are vectors, compositions and methods for treating and diagnosing breast cancer. For example, the present disclosure provides a vector for the expression of a therapeutic protein, wherein the vector comprises a microRNA binding domain (MBD) that facilitates the expression of the therapeutic protein in breast cancer cells and inhibits the expression of the therapeutic protein in non-breast cancer cells.

Accordingly, in one aspect, provided herein is a vector comprising (a) a first deoxyribonucleic acid (DNA) sequence comprising a transgene; and (b) a second deoxyribonucleic (DNA) acid sequence comprising a microRNA binding domain (MBD); wherein the MBD comprises one or more microRNA binding sites (MBSs), wherein each MBS is specific for a microRNA that is present in a non-breast cancer cell and is not present or is downregulated in a breast cancer cell, wherein the one or more MBSs are specific for one or more microRNAs selected from one of the combinations presented in Tables 1-4.

In another aspect, the vector comprises (a) a first deoxyribonucleic acid (DNA) sequence comprising a transgene; and (b) a second deoxyribonucleic acid (DNA) sequence comprising a microRNA binding domain (MBD); wherein the MBD comprises one or more microRNA binding sites (MBSs), wherein each MBS is specific for a microRNA that is present in a non-breast cancer cell and is not present or is downregulated in an early stage breast cancer cell.

In yet another aspect, the vector comprises (a) a first deoxyribonucleic acid (DNA) sequence comprising a transgene; and (b) a second deoxyribonucleic acid (DNA) sequence comprising a microRNA binding domain (MBD); wherein the MBD comprises one or more microRNA binding sites (MBSs), wherein each MBS is specific for a microRNA that is present in a non-breast cancer cell and is not present or is downregulated in a late stage breast cancer cell.

The present disclosure also provides compositions comprising the vectors and methods of using the vectors for treating and/or diagnosing breast cancer.

### BRIEF DESCRIPTION OF THE DRAWINGS

FIG. 1 depicts an exemplary transgene expression construct according to the disclosure.

FIG. 2A depicts an exemplary template vector that can be used to generate the miRNA-regulated expression vector according to the disclosure.

FIG. 2B depicts an exemplary miRNA-regulated expression vector according to the disclosure known as pSUPON-TGG.

FIG. 3 is a bar graph showing the percentage of expression of GFP in early stage breast cancer cells (MCF7) and healthy breast cells (MCF10A) transfected with an miRNA-regulated expression vector containing the GFP transgene.

FIG. 4 is a bar graph showing the expression of GFP in healthy breast cells (MCF10A) transfected with a control GFP vector (where the GFP transgene is not regulated by miRNAs) and an miRNA-regulated GFP expression vector.

FIG. 5 is a bar graph showing the expression of GFP in early stage breast cancer cells (MCF7) transfected with a control GFP vector and an miRNA-regulated GFP expression vector.

FIG. 6 is a bar graph showing the expression of GFP in late stage breast cancer cells (BT549) transfected with a control GFP vector and an miRNA-regulated GFP expression vector.

FIG. 7A is a bar graph showing the normalized fold difference of GFP expression over time between healthy breast cells, MCF10A, and early stage breast cancer cells, MCF7.

FIG. 7B is a bar graph showing the normalized fold difference of GFP expression over time between healthy breast cells, MCF10A, and late stage breast cancer cells, BT549.

FIG. 8A is a graph showing cell count over time in healthy breast cells, MCF10A, treated with the vector depicted in FIG. 2B.

FIG. 8B is a graph showing cell count over time in cancerous breast cells, BT549, treated with the vector depicted in FIG. 2B.

FIG. 9 depicts an exemplary HSVtk vector, namely, the pSELECT-zeo-HSV1tk vector, from Invivogen that may be used to clone the HSVtk gene into the miRNA-regulated vectors of the disclosure.

FIG. 10 depicts an exemplary vector, pEGG-SUPON, that can be further modified to prepare miRNA-regulated vectors of the disclosure.

FIG. 11 depicts an exemplary miRNA-regulated vector of the disclosure.

FIG. 12 depicts an exemplary vector, pSV-TGG-SUPON, that can be further modified to prepare miRNA-regulated vectors of the disclosure.

FIG. 13A is a graph showing cell count over time in cancerous breast cells, BT549, untreated or treated with ganciclovir and transfected with a miR-205-3p regulated vector with the SV40 promoter.

FIG. 13B is a graph showing cell count over time in healthy breast cells, MCF10A, untreated or treated with ganciclovir and transfected with a miR-205-3p regulated vector with the SV40 promoter.

FIG. 13C is a graph showing cell count over time in cancerous breast cells, BT549, untreated or treated with ganciclovir and transfected with a miR-205-3p regulated vector with the CAG promoter.

FIG. 13D is a graph showing cell count over time in healthy breast cells, MCF10A, untreated or treated with ganciclovir and transfected with a miR-205-3p regulated vector with the CAG promoter.

FIG. 14A is a graph showing cell count over time in cancerous breast cells, BT549, untreated or treated with ganciclovir and transfected with a miR-205-5p regulated vector with the SV40 promoter.

FIG. 14B is a graph showing cell count over time in healthy breast cells, MCF10A, untreated or treated with ganciclovir and transfected with a miR-205-5p regulated vector with the SV40 promoter.

FIG. 14C is a graph showing cell count over time in cancerous breast cells, BT549, untreated or treated with ganciclovir and transfected with a miR-205-5p regulated vector with the CAG promoter.

FIG. 14D is a graph showing cell count over time in healthy breast cells, MCF10A, untreated or treated with ganciclovir and transfected with a miR-205-5p regulated vector with the CAG promoter.

FIG. 15A is a graph showing cell count over time in cancerous breast cells, BT549, untreated or treated with ganciclovir and transfected with a miR-200C regulated vector with the SV40 promoter.

FIG. 15B is a graph showing cell count over time in healthy breast cells, MCF10A, untreated or treated with ganciclovir and transfected with a miR-200C regulated vector with the SV40 promoter.

FIG. 15C is a graph showing cell count over time in cancerous breast cells, BT549, untreated or treated with ganciclovir and transfected with a miR-200C regulated vector with the CAG promoter.

FIG. 15D is a graph showing cell count over time in healthy breast cells, MCF10A, untreated or treated with ganciclovir and transfected with a miR-200C regulated vector with the CAG promoter.

FIG. 16A is a graph showing cell count over time in cancerous breast cells, BT549, treated with ganciclovir and transfected with a positive control vector comprising the SV40 promoter.

FIG. 16B is a graph showing cell count over time in healthy breast cells, MCF10A, treated with ganciclovir and transfected with a positive control vector comprising the SV40 promoter.

FIG. 16C is a graph showing cell count over time in cancerous breast cells, BT549, treated with ganciclovir and transfected with a positive control vector comprising the CAG promoter.

FIG. 16D is a graph showing cell count over time in healthy breast cells, MCF10A, treated with ganciclovir and transfected with a positive control vector comprising the CAG promoter.

FIG. 17A shows normalized fold differences in the cell killing between MCF10A and BT549 transfected with a miR-205-3p-regulated vector with the SV40 promoter.

FIG. 17B shows normalized fold differences in the cell killing between MCF10A and BT549 transfected with a miR-205-5p-regulated vector with the SV40 promoter.

FIG. 17C shows normalized fold differences in the cell killing between MCF10A and BT549 transfected with a miR-200C-3p-regulated vector with the SV40 promoter.

FIG. 18A shows normalized fold differences in the cell killing between MCF10A and BT549 transfected with a miR-205-3p-regulated vector with the CAG promoter.

FIG. 18B shows normalized fold differences in the cell killing between MCF10A and BT549 transfected with a miR-205-5p-regulated vector with the CAG promoter.

FIG. 18C shows normalized fold differences in the cell killing between MCF10A and BT549 transfected with a miR-200C-3p-regulated vector with the CAG promoter.

### DETAILED DESCRIPTION OF THE DISCLOSURE

Provided herein are vectors, compositions and methods for treating and diagnosing breast cancer. In particular, the present disclosure provides a vector for the expression of a therapeutic protein, wherein the vector comprises a microRNA binding domain (MBD) that facilitates the expression of the therapeutic protein in breast cancer cells and inhibits the expression of the therapeutic protein in non-breast cancer cells. The present disclosure also provides compositions comprising the vectors and methods of using the vectors for treating and/or diagnosing breast cancer.

Before describing certain embodiments in detail, it is to be understood that this disclosure is not limited to particular compositions or biological systems, which can vary. It is also to be understood that the terminology used herein is for the purpose of describing particular illustrative embodiments only, and is not intended to be limiting. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the disclosure and how to make and use them. The scope and meaning of any use of a term will be apparent from the specific context in which the term is used. As such, the definitions set forth herein are intended to provide illustrative guidance in ascertaining particular embodiments of the disclosure, without limitation to particular compositions or biological systems.

As used in the present disclosure and the appended claims, the singular forms “a,” “an” and “the” include plural references unless the content clearly dictates otherwise.

Throughout the present disclosure and the appended claims, unless the context requires otherwise, the word “comprise”, or variations such as “comprises” or “comprising”, will be understood to imply the inclusion of a stated element or group of elements but not the exclusion of any other element or group of elements.

As used herein, the terms “microRNA,” “miRNA,” and “miR” are used interchangeably and refer to a non-coding RNA that is about 20 to 35 nucleotides long and that post-transcriptionally regulates the cleavage of a target mRNA or represses the translation of the target mRNA. Throughout this disclosure, the effect of binding of miRNAs to the miRNA binding sites (MBSs) of a microRNA binding domain (MBD) is described. In so describing, the effects include preventing, and/or inhibiting/repressing the cleavage of the transgene mRNA and/or inhibition of the translation of the transgene mRNA. A specific miRNA recited herein, for example, miR-205, miR-100, miR-423, let-7b, and so on, encompasses any miRNA sequence that shares at least the seed sequence of that miRNA's family (including both -5p and -3p forms) as per the miRBase database version 22.1, October 2018, and any established isoforms of the family members, including those with up to 2 base pairs of seed shifting either in the 5′ direction or 3′ direction of the miRNA.

The term “early stage breast cancer” as used herein refers to cancer that has not spread beyond the breast or axillary lymph nodes. This includes ductal carcinoma in situ and stage IA, stage IB, stage IIA, stage IIB and stage IIIA breast cancers as defined by the American Joint Committee on Cancer (AJCC) in the AJCC Cancer Staging Manual, 7th Edition.

The term “late stage breast cancer” as used herein refers to cancer originating in the breast that is far along in its growth and has spread beyond the axillary lymph nodes and other areas in the body. This includes stage IIIB, stage IIIC and stage IV breast cancer as defined by the American Joint Committee on Cancer (AJCC) in the AJCC Cancer Staging Manual, 7th Edition.

The term “non-breast cancer cell” as used herein encompasses a heathy breast cell, a breast cell that is non-cancerous, and a cell from any other organ or tissue. The term “non early-stage breast cancer cell” as used herein encompasses a heathy breast cell, a breast cell that is non-cancerous, a late stage breast cancer cell, and a cell from any other organ or tissue. The term “non late-stage breast cancer cell” as used herein encompasses a heathy breast cell, a breast cell that is non-cancerous, an early stage breast cancer cell, and a cell from any other organ or tissue. The terms “healthy” and “normal” are used interchangeably throughout the disclosure.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery.

### Vectors

The present disclosure provides a vector comprising a transgene and a binding domain for microRNAs. This microRNA binding domain (MBD) comprises one or more microRNA binding sites (MBSs), wherein each MBS is specific for a microRNA that is endogenously expressed in a non-breast cancer cell and is not expressed or is downregulated in a breast cancer cell. In the presence of specific microRNAs for which the MBSs are present in the vector, the transgene is not expressed or is minimally expressed. Without being bound by theory, the microRNAs can bind to the MBSs and inhibit or prevent the translation of the transgene mRNA (e.g. by inducing cleavage of the transgene mRNA or repressing the translation of the transgene mRNA). On the other hand, the transgene can be expressed in cells where the specific microRNAs are not present or are downregulated. For example, the transgene can be expressed in breast cancer cells where the specific microRNAs are not present or are downregulated.

In some embodiments, the vector comprises a first deoxyribonucleic acid (DNA) sequence comprising a transgene and a second DNA sequence comprising a MBD, wherein the MBD comprises one or more MBSs, wherein each MBS is specific for a microRNA that is present in a non-breast cancer cell and is not present or is downregulated in a breast cancer cell. As used herein, a vector comprising at least a first DNA sequence comprising a transgene, and a second DNA sequence comprising a MBD is referred to as a “miRNA-regulated expression vector” or “miRNA-regulated vector”.

In some embodiments, the second DNA sequence comprising a MBD is linked to the first DNA sequence comprising a transgene in tandem, i.e., there is no overlap between the first and the second DNA sequences. In other embodiments, the second DNA sequence comprising a MBD is located within the first DNA sequence comprising a transgene.

In some embodiments, the breast cancer cell is an early stage breast cancer cell. Accordingly, in such embodiments, the vector comprises a first DNA sequence comprising a transgene; and a second DNA sequence comprising a MBD; wherein the MBD comprises one or more MBSs, wherein each MBS is specific for a microRNA that is present in a non early-stage breast cancer cell and is not present or is downregulated in an early stage breast cancer cell.

In other embodiments, the breast cancer cell is a late stage breast cancer cell. Accordingly, in such embodiments, the vector comprises a first DNA sequence comprising a transgene; and a second DNA sequence comprising a MBD; wherein the MBD comprises one or more MBSs, wherein each MBS is specific for a microRNA that is present in a non late-stage breast cancer cell and is not present or is downregulated in a late stage breast cancer cell.

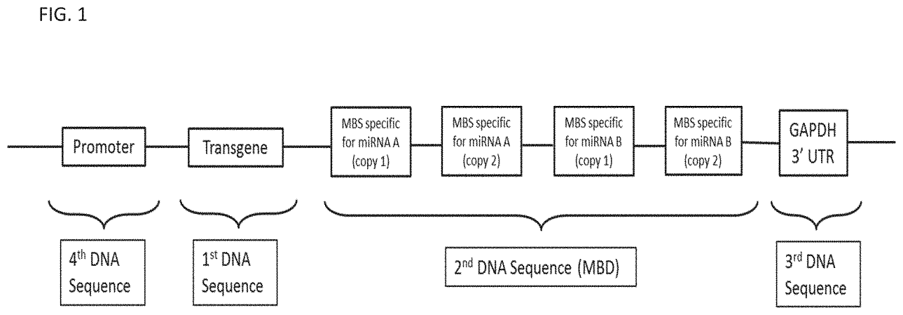

FIG. 1 shows an exemplary transgene expression construct that is present in a vector of the present disclosure.

As provided herein and illustrated in FIG. 1, the MBD comprises MBSs. In some embodiments, the MBD can comprise 1 to 12 MBSs, e.g. the MBD comprises MBSs that are specific for 1 to 12 microRNAs. In various embodiments, the MBD may comprise about 1-12, about 1-10, about 1-8, about 2-12, about 2-10, about 2-8, about 2-6, about 2-5, about 3-12, about 3-12, about 3-10, about 3-8, about 3-6, about 4-12, about 4-10, about 4-8, about 4-6, about 5-10, or about 5-8 MBSs. For example, FIG. 1 exemplifies a vector where the MBD comprises 2 MBSs; each MBS being specific for a different microRNA.

In some embodiments, the MBSs could be specific for the ˜6 to ˜8-nucleotide “seed” sequence at the 5′ end of a miRNA. In some embodiments, the MBSs could be specific for other regions of miRNAs such as the sequence at the 3′ end of a miRNA. In yet some other embodiments, the MBSs could be specific for a sequence of a miRNA that forms the central loop in the miRNA:mRNA duplexes. In some embodiments, the MBSs could be specific for combinations of these features.

Multiple copies of each MBS may be present in the MBD. In various embodiments, 1-12, 2-10, 4-8, or 3-6 copies of each MBS may be present in the MBD. In some embodiments, the MBD comprises at least 2 copies of each MBS. In some other embodiments, the MBD comprises 3, 4, 5, or 6 copies of each MBS. When the MBD comprises more than one MBS and multiple copies of each MBS are present, the multiple copies of each MBS may be present as a single cluster or the multiple copies may be scattered throughout the MBD, i.e. one or more copies of each MBS may alternate with one or more copies of other MBSs. For example, the exemplary expression construct shown in FIG. 1 comprises 2 MBSs in the MBD and 2 copies of each MBS are present in the MBD.

The length of each copy of an MBS can be selected based on desired binding aspects. In some embodiments, the length of each copy of an MBS may range from about 6 to 33 nucleotides. For example, the length of each copy of a MBS could be about 6 to about 33 nucleotides, about 6 to 30 nucleotides, about 6 to 27 nucleotides, about 6 to 25 nucleotides, about 6 to 23 nucleotides, about 6 to 20 nucleotides, about 6 to 18 nucleotides, about 6 to 15 nucleotides, about 6 to 13 nucleotides, about 6 to 11 nucleotides, or about 6 to 8 nucleotides. In some embodiments, the length of each copy of a MBS is about 6 to 13 nucleotides.

As provided herein, an MBD can comprise multiple MBSs that are specific for different microRNAs. Also contemplated herein, an MBD can comprise multiple MBSs that are specific for the same microRNA, but bind to different regions of the microRNA. Also contemplated herein, an MBD can comprise multiple copies of the same MBS, or MBSs with only slight variations in between.

Multiple copies of each MBS may be separated from each other by spacer sequences that are about 5 to 50 nucleotides long. For example the spacer sequences could be about 5 to 45, about 5 to 40, about 5 to 35, about 5 to 30, about 5 to 25, about 5 to 20, about 5 to 15, about 10 to 50, about 10 to 45, about 10 to 40, about 10 to 35, about 10 to 30, about 10 to 25, about 10 to 20, about 10 to 15, about 15 to 50, about 15 to 45, about 15 to 40, about 15 to 35, about 15 to 30, about 15 to 25, about 15 to 20 nucleotides long. The individual MBSs may be separated from each other by about the same number of nucleotides.

In various embodiments, the second DNA sequence comprising the MBD can be located on either the 3′ or the 5′ side of the first DNA sequence. For example, in the exemplary expression construct shown in FIG. 1, the second DNA sequence is on the 3′ side of the first DNA sequence.

As provided herein, the transgene of the described miRNA-regulated vector encodes a therapeutic protein or a reporter protein. The first DNA sequence comprising a transgene comprises the coding sequence of the protein encoded by the transgene, i.e., the DNA sequence does not contain any introns, unless the introns are determined to be required for the proper transcription of the transgene, proper functioning of the therapeutic protein, regulation of the transgene expression by miRNAs or any other aspect of the expression of the transgene. The first DNA sequence comprises a terminator sequence that marks the end of the coding sequence and mediates transcription termination.

In some embodiments, the miRNA-regulated vectors may comprise additional components that mediate further repression of the transgene mRNA. For example, in some embodiments, 3′ UTRs that provide a high translational efficiency to an mRNA may be included in the vectors. In some embodiments, a translational efficiency can be quantified as the ratio of ribosome protected fragments (RPF) to the abundance of ribonucleic acids (RNA) (Cottrell et al., Sci Rep. 2017 Nov. 2; 7(1):14884). In some embodiments, the present disclosure provides vectors that comprise 3′ UTRs that provide a translational efficiency of about −0.25 to about −0.8, about −0.28 to about −0.8, about −0.3 to about −0.8, about −0.25 to about −0.75, −0.25 to about −0.7, about −0.25 to about −0.6, about −0.3 to about −0.75, including values and ranges therebetween. In these embodiments, the vector comprises a third DNA sequence that comprises one or more 3′ UTRs of one or more genes.

The first, second, and third DNA sequences of the miRNA-regulated vectors can be linked in any order. For example, in some embodiments, the first, second, and third DNA sequences are linked such that the second DNA sequence is 3′ of the first DNA sequence, and the third DNA sequence is 3′ of the second DNA sequence. In other embodiments, the second DNA sequence is 5′ of the first DNA sequence, and the third DNA sequence is 3′ of the first DNA sequence. In some other embodiments, the second DNA sequence is within the first DNA sequence and the third DNA sequence is 3′ of the first DNA sequence.

In some embodiments, the third DNA sequence comprises 3′ UTRs of one or more housekeeping genes, e.g., GAPDH, or cytoskeleton genes, e.g., α-tubulin, and β-tubulin. In some other embodiments, the third DNA sequence comprises 3′ UTRs of one or more genes selected from the group consisting of: Rp132, HSP70a, and CrebA.

In some embodiments, the miRNA-regulated vector comprises a fourth DNA sequence, 5′ of the first DNA sequence, wherein the fourth DNA sequence comprises a promoter, optionally with an enhancer. Accordingly, in such embodiments, the first DNA sequence comprising a transgene, the second DNA sequence comprising a MBD, and the third DNA sequence comprising 3′ UTRs of one or more genes, if present, are under the control of the same promoter (under the control of an identical promoter, and enhancer, if present).

In some embodiments, the promoter is specifically expressed in breast cells. In some embodiments, the promoter is selected from the group consisting of: SV40, CMV, EF1α, PGK1, Ubc, human β actin, CAG, TRE, UAS, Ac5, polyhedrin, CaMKIIa, Gal 1/10, TEF1, GDS, ADH1, CaMV35S, Ubi, H1, and U6.

The second DNA sequence comprising the MBD may start from about 1 to 50 nucleotides after the last nucleotide of the stop codon of the transgene. In various embodiments, the second DNA sequence may start from about 1 to 45, about 1 to 40, about 1 to 35, about 1 to 30, about 1 to 25, about 1 to 20, about 1 to 15, or about 1 to 10 nucleotides after the last nucleotide of the stop codon of the transgene.

In some embodiments, the miRNA-regulated vector may comprise a fifth DNA sequence containing a repressor element that is 5′ of the first DNA sequence. This repressor element facilitates further repression of the expression of the transgene. In some embodiments, the fifth DNA sequence encodes a hemagglutinin-A epitope.

The first DNA sequence, the second DNA sequence, the third DNA sequence, the fourth DNA sequence, and/or the fifth DNA sequence together may be referred to as an expression cassette or an expression construct. The sequences can be linked in any order.

As provided herein, the transgene present in the miRNA-regulated vectors of the present disclosure can encode a therapeutic protein or a reporter protein.

As provided herein, a therapeutic protein is any protein that inhibits the proliferation and/or metastasis of breast cancer cells. Examples of therapeutic proteins include, but are not limited to, apoptosis inducers, growth regulators, tumor suppressors, ion channels, cell-surface or internal antigens, or any protein mutated in breast cancer cells (e.g. BRCA1, BRCA2, etc.). In certain embodiments, the therapeutic protein is an apoptosis inducing protein, such as a caspase, thymidine kinase, e.g., Herpes Simplex Virus thymidine kinase (HSV-tk), a granzyme, an exotoxin, or a proapoptotic member of the Bcl-2 family. Expression of the HSV-tk mediates phosphorylation of the prodrug gancyclovir (GCV), thus inhibiting DNA replication in rapidly dividing cancer cells. As phosphorylated GCV is also toxic in normal cells, tumor cell-specific expression of HSVtk is required and can be accomplished using the vectors of the present disclosure.

As provided herein, the transgene may be a reporter gene encoding for a reporter protein, e.g. useful for in vitro, in vivo, or ex vivo diagnostics or medical imaging. In some embodiments, the reporter transgene encodes a fluorescent protein or a bioluminescent protein. For example, the transgene may encode a fluorescent protein selected from the group consisting of: green fluorescent protein, cyan fluorescent protein, yellow fluorescent protein, red fluorescent protein, far-red fluorescent protein, orange fluorescent protein, and ultraviolet-excitable green fluorescent protein. These reporter proteins are known and are summarized by Shaner et al., Nat Methods., 2005 December; 2(12):905-9.

In some embodiments, the reporter transgene encodes for a bioluminescent protein including a firefly luciferase such as Renilla luciferase.

In some embodiments, the miRNA-regulated vectors may comprise an additional second set of DNA sequences comprising a second transgene that is under the control of a second MBD containing one or more MBSs as described above.

The miRNA-regulated vectors can be viral DNA vectors or non-viral DNA vectors. Examples of viral vectors include retroviral vectors, lentiviral vectors, adenoviral vectors, adeno-associated viral vectors, and herpes simplex virus vectors. Non-viral vectors include plasmids and cosmids.

The miRNA-regulated vectors of the present disclosure are used to express a transgene specifically in breast cancer cells. A transgene encodes a protein of interest, e.g., a therapeutic protein or a reporter protein. The expression of the protein of interest is regulated by endogenously expressed miRNAs. The MBSs present in the MBD are specific for one or more miRNAs that are present in non-breast cancer cells and are absent or are down-regulated in breast cancer cells. Upon transcription of the first and second DNA sequences containing the transgene and the MBD respectively, target miRNAs present in non-breast cancer cells are intended to bind to their corresponding MBSs present on the MBD of the transgene mRNA and inhibit the translation of the transgene mRNA thereby inhibiting the expression of the protein of interest. In breast cancer cells, the transgene mRNA is intended to be translated and the protein of interest would be expressed since target miRNAs are absent or are down-regulated in breast cancer cells.

In some embodiments, multiple vectors can be utilized to enhance the selective expression of the transgene by creating an “artificial pathway.” In these embodiments, vectors would comprise additional regulatory elements to provide an enhanced effect. For example, in some embodiments, two vectors can be provided where one of the vectors comprises a DNA sequence comprising a transgene encoding a transcriptional repressor (e.g. Lad) and MBSs for one or more miRNAs expressed in a breast cancer cell but are not present or are downregulated in a healthy cell (e.g. miRNAs listed in Table 5) and the other vector comprises a DNA sequence comprising a transgene encoding a therapeutic protein, a binding sequence (e.g. LacO sites) for the transcriptional repressor upstream of the transgene, and MBSs for one or more miRNAs expressed in a healthy cell but are not present or are downregulated in a breast cancer cell (e.g. miRNAs listed in Tables 1-4). The transgene encoding the transcriptional repressor and the transgene encoding the therapeutic protein can be expressed using a tissue-specific promoter (e.g. MMTV, WAP, etc.) or a constitutive promoter (e.g CAG, CMV, EF1-alpha, etc.). The transcriptional repressor would be expressed in healthy cells but its expression would be down-regulated in cancer cells whereas the expression of the therapeutic protein in the cancer cells would be controlled by two variables: the extent of Lad expression in the cancer cells (down-regulated in cancer cells but highly expressed in healthy cells) and the level of expression of one or more miRNAs from Tables 1-4 in the cancer cells. In this example, the two vectors would provide enhanced specificity of expression of the therapeutic protein in the cancer cells.

### Exemplary Embodiments

In various embodiments, the MBSs present in the MBD of the vectors of the present disclosure are specific for one or more miRNAs selected from one of the combinations listed in Tables 1-5. Each microRNA listed in the tables below encompasses both the 3p and 5p forms of that microRNA. For example, miR-629 encompasses miR-629-3p and miR-629-5p and so on.

TABLE 1

| MBSs Combinations | MBSs in the MBD |
|---|---|
| Combination 1 | miR-629, miR-200C, miR-203A, miR-4760, miR-429, miR-95, and/or |
| | miR-489 |
| Combination 2 | miR-452, miR-224, miR-100, miR-31, and/or miR-10A |
| Combination 3 | miR-224, miR-577, miR-452, miR-221, miR-100, miR-205, and/or |
| | miR-31 |
| Combination 4 | miR-205, miR-200C, and/or miR-510 |
| Combination 5 | miR-200C and miR-203c |
| Combination 6 | miR-452, miR-224, miR-100, and/or miR-31 |
| Combination 7 | miR-100, miR-138, miR-221, miR-222, and/or miR- 205 |
| Combination 8 | miR-205 and/or miR-34c |
| Combination 9 | miR-205, miR-34c, miR-203c, and/or miR-200C |
| Combination 10 | miR-629, miR-200C, miR-203A, miR-4760, miR-429, miR-95, miR- |
| | 489, miR-205, miR-510, miR-34c, and/or miR-203c |
| Combination 11 | miR-452, miR-224, miR-100, miR-31, miR-10A, miR-577, miR-221, |
| | miR-205, and/or miR-34c |
| Combination 12 | miR-629, miR-200C, miR-203A, miR-4760, miR-429, miR-95, miR- |
| | 489, miR-452, miR-224, miR-100, miR-31, miR-10A, miR-577, miR- |
| | 221, miR-205, miR-510, miR-138, miR-222, miR-205, miR-34c, |
| | and/or miR-203c |

Table 2 shows miRNAs upregulated or expressed abundantly in healthy cells (e.g. CCD1070sk or MCF10A) but down-regulated in cancer cells (e.g. BT549 or MCF7). The vectors of the present disclosure can comprise MBSs for these microRNAs to regulate the expression of the transgene in breast cancer cells.

TABLE 2

| | |
|---|---|
| Combination 13 | let-7b, miR-423, miR-423, miR-34c, miR-34a, and/or miR-296 |
| Combination 14 | miR-200c, miR-205, miR-92a, miR-20a, miR-378a, miR-19b, miR-17, |
| | miR-183, miR-92b, miR-181b, miR-19a, miR-18a, miR-708, miR-92a-1, |
| | miR-584, miR-514a, miR-944, and/or miR-205 |
| Combination 15 | miR-152, miR-455, miR-218, miR-143, miR-889, miR-138, miR-382, miR- |
| | 199a, miR-487b, miR-134, miR-199a, miR-369, miR-494, miR-381, miR- |
| | 10b, miR-145, miR-410, miR-199b, miR-329, miR-654, miR-376c, miR- |
| | 409, miR-199b, miR-758, miR-369, miR-495, miR-145, miR-379, miR- |
| | 323a, miR-377, miR-411, miR-487a, miR-539, miR-323b, miR-380, miR- |
| | 412, miR-655, miR-1185-1, miR-127, miR-337, miR-382, miR-485, miR- |
| | 654, miR-143, miR-370, miR-376a, miR-377, miR-432, miR-485, miR-543, |
| | miR-10b, miR-1185-2, miR-136, miR-136, miR-154, miR-154, miR-214, |
| | miR-214, miR-299, miR-299, miR-337, miR-431, miR-433, miR-490, miR- |
| | 490, miR-493, miR-493, miR-539, miR-656, and/or miR-665 |
| Combination 16 | let-7b, miR-423, miR-423, miR-34c, miR-34a, and miR-296, miR-200c, |
| | miR-205, miR-92a, miR-20a, miR-378a, miR-19b, miR-17, miR-183, miR- |
| | 92b, miR-181b, miR-19a, miR-18a, miR-708, miR-92a-1, miR-584, miR- |
| | 514a, miR-944, and miR-205, miR-152, miR-455, miR-218, miR-143, miR- |
| | 889, miR-138, miR-382, miR-199a, miR-487b, miR-134, miR-199a, miR- |
| | 369, miR-494, miR-381, miR-10b, miR-145, miR-410, miR-199b, miR- |
| | 329, miR-654, miR-376c, miR-409, miR-199b, miR-758, miR-369, miR- |
| | 495, miR-145, miR-379, miR-323a, miR-377, miR-411, miR-487a, miR- |
| | 539, miR-323b, miR-380, miR-412, miR-655, miR-1185-1, miR-127, miR- |
| | 337, miR-382, miR-485, miR-654, miR-143, miR-370, miR-376a, miR-377, |
| | miR-432, miR-485, miR-543, miR-10b, miR-1185-2, miR-136, miR-136, |
| | miR-154, miR-154, miR-214, miR-214, miR-299, miR-299, miR-337, miR- |
| | 431, miR-433, miR-490, miR-490, miR-493, miR-493, miR-539, miR-656, |
| | and/or miR-665 |

Table 3 shows miRNAs upregulated in early stage breast cancer cells (e.g. MCF7) but down-regulated in healthy cells (e.g. CCD1070sk or MCF10A) or late stage breast cancer cells (e.g. BT549). The vectors of the present disclosure can comprise MBSs for these microRNAs to regulate the expression of the transgene in late stage breast cancer cells.

TABLE 3

| Combination 17 | miR-125a, miR-99b, miR-182, miR-93, miR-148b, miR-425, miR-30d, |
|---|---|
| | miR-26b, miR-484, miR-96, miR-185, miR-25, miR-203a, miR-454, miR- |
| | 7, miR-23b, miR-342, miR-421, miR-106b, miR-141, miR-95, miR-345, |
| | miR-429, miR-542, miR-200b, miR-200a, miR-489, miR-618, and/or miR- |
| | 653 |

Table 4 shows miRNAs upregulated in late stage breast cancer cells (e.g. BT549) but down-regulated in healthy cells (e.g. CCD1070sk or MCF10A) or early stage breast cancer cells (e.g. MCF7). The vectors of the present disclosure can comprise MBSs for these microRNAs to regulate the expression of the transgene in early stage breast cancer cells.

TABLE 4

| | |
|---|---|
| Combination 18 | miR-221, miR-100, miR-22, miR-29a, miR-320a, miR-222, miR-31, |
| | miR-30c, miR-135b, miR-362, miR-146a, miR-221, miR-10a, miR-30a, |
| | miR-30a, miR-486, miR-582, miR-196a, miR-1271, miR-379, miR-409, |
| | and/or miR-411 |

Table 5 shows miRNAs upregulated in breast cancer cells (BT549 or MCF7) but down-regulated in healthy cells (CCD1070sk or MCF10A). The vectors of the present disclosure can comprise MBSs for these microRNAs to regulate the expression of the transgene in healthy cells.

TABLE 5

| | |
|---|---|
| Combination 19 | miR-155, let-7i, miR-27b, miR-191, miR-27a, miR-99a, miR-151a, miR- |
| | 450b, and/or miR-450a |

For example, in some embodiments, the vector comprises a first DNA sequence comprising a transgene and a second DNA sequence comprising a MBD, wherein the MBD comprises one or more MBSs, wherein each MBS is specific for a microRNA that is present in a non-breast cancer cell and is not present or is downregulated in a breast cancer cell, and wherein the one or more MBSs are specific for one or more microRNAs selected from miR-629, miR-200C, miR-203A, miR-4760, miR-429, miR-95, and miR-489 (Combination 1 of Table 1). In some embodiments, the MBD comprises at least two MBSs, wherein the MBSs are specific for at least two microRNAs present in a non-breast cancer cell and not present or downregulated in a breast cancer cell. In some embodiments, the MBD comprises at least three MBSs, wherein the MBSs are specific for at least three microRNAs present in a non-breast cancer cell and not present or downregulated in a breast cancer cell. In some embodiments, the MBD comprises at least four or at least five MBSs, wherein the MBSs are specific for at least four or five microRNAs present in a non-breast cancer cell and not present or downregulated in a breast cancer cell. In some embodiments, the vectors of the present disclosure comprise one or more MBSs that are specific for one or more microRNAs selected from one of the Combinations 1-19 of Tables 1-5. In some embodiments, the vectors of the present disclosure comprise at least two MBSs specific for at least two microRNAs selected from one of the Combinations 1-19 of Tables 1-5. In some embodiments, the vectors of the present disclosure comprise at least three MBSs specific for at least three microRNAs selected from one of the Combinations 1-19 of Tables 1-5. In some embodiments, the vectors of the present disclosure comprise at least four or at least five MBSs specific for at least four or at least five microRNAs selected from one of the Combinations 1-19 of Tables 1-5.

The inventors have found specific microRNAs that are absent or are down-regulated in breast cancer cells. For example, the inventors have found that miR-629, miR-200C, miR-203A, miR-4760, miR-429, miR-95, and miR-489 are absent or are down-regulated in late stage breast cancer cells, but are not down-regulated in early stage breast cancer cells. Accordingly, in some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-629, miR-200C, miR-203A, miR-4760, miR-429, miR-95, miR-489, and combinations thereof (Combination 1). Using this vector, the transgene can be expressed in a late stage breast cancer cell.

The inventors have found that miR-452, miR-224, miR-100, miR-31, and miR-10A are absent or are down-regulated in early stage breast cancer cells, but are not down-regulated in late stage breast cancer cells. Accordingly, in some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-452, miR-224, miR-100, miR-31, miR-10A, and combinations thereof (Combination 2). Using this vector, the transgene can be expressed in an early stage breast cancer cell.

The inventors have found that miR-224, miR-577, miR-452, miR-221, miR-100, miR-205, and miR-31 are absent or are down-regulated in early stage breast cancer cells, but are not down-regulated in normal breast cells. Accordingly, in some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-224, miR-577, miR-452, miR-221, miR-100, miR-205, miR-31, and combinations thereof (Combination 3). Using this vector, the transgene can be expressed in an early stage breast cancer cell.

The inventors have found that miR-205, miR-200C, and miR-510 are absent or are down-regulated in late stage breast cancer cells, but are not down-regulated in normal breast cells. Accordingly, in some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-205, miR-200C, miR-510, and combinations thereof (Combination 4). Using this vector, the transgene can be expressed in a late stage breast cancer cell.

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-200C, miR-203C, and combinations thereof (Combination 5). Using this vector, the transgene can be expressed in a late stage breast cancer cell.

In some other embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-452, miR-224, miR-100, miR-31, and combinations thereof (Combination 6). Using this vector, the transgene can be expressed in an early stage breast cancer cell.

Studies have shown that miR-100, miR-138, miR-221, miR-222, and miR-205 are down-regulated in breast cancer cells. Accordingly, in some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-100, miR-138, miR-221, miR-222, miR-205, and combinations thereof (Combination 7). Using this vector, the transgene can be expressed in a breast cancer cell.

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-205, miR-34C, and combinations thereof (Combination 8). Using this vector, the transgene can be expressed in a breast cancer cell.

In other embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-205, miR-34C, miR-203C, miR-200C, and combinations thereof (Combination 9). Using this vector, the transgene can be expressed in a late stage breast cancer cell.

In some other embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-629, miR-200C, miR-203A, miR-4760, miR-429, miR-95, miR-489, miR-205, miR-510, miR-34C, miR-203C, and combinations thereof (Combination 10). Using this vector, the transgene can be expressed in a late stage breast cancer cell.

In yet some other embodiments, the vector comprises a first DNA sequence comprising a transgene and a second DNA sequence comprising a MBD, wherein the MBD comprises one or more MBSs, wherein the one or more MBSs are specific for one or more microRNAs selected from a group consisting of: miR-452, miR-224, miR-100, miR-31, miR-10A, miR-577, miR-221, miR-205, miR-34C, and combinations thereof (Combination 11). Using this vector, the transgene can be expressed in an early stage breast cancer cell.

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from a group consisting of: miR-629, miR-200C, miR-203A, miR-4760, miR-429, miR-95, miR-489, miR-452, miR-224, miR-100, miR-31, miR-10A, miR-577, miR-221, miR-205, miR-510, miR-138, miR-222, miR-205, miR-34C, miR-203C, and combinations thereof (Combination 12).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: let-7b, miR-423, miR-423, miR-34c, miR-34a, miR-296, and combinations thereof (Combination 13).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-200, miR-205, miR-92a, miR-20a, miR-378a, miR-19b, miR-17, miR-183, miR-92b, miR-181b, miR-19a, miR-18a, miR-708, miR-92a-1, miR-584, miR-514a, miR-944, miR-205, and combinations thereof (Combination 14).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-152, miR-455, miR-218, miR-143, miR-889, miR-138, miR-382, miR-199a, miR-487b, miR-134, miR-199a, miR-369, miR-494, miR-381, miR-10b, miR-145, miR-410, miR-199b, miR-329, miR-654, miR-376c, miR-409, miR-199b, miR-758, miR-369, miR-495, miR-145, miR-379, miR-323a, miR-377, miR-411, miR-487a, miR-539, miR-323b, miR-380, miR-412, miR-655, miR-1185-1, miR-127, miR-337, miR-382, miR-485, miR-654, miR-143, miR-370, miR-376a, miR-377, miR-432, miR-485, miR-543, miR-10b, miR-1185-2, miR-136, miR-136, miR-154, miR-154, miR-214, miR-214, miR-299, miR-299, miR-337, miR-431, miR-433, miR-490, miR-490, miR-493, miR-493, miR-539, miR-656, miR-665, and combinations thereof (Combination 15).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: let-7b, miR-423, miR-423, miR-34c, miR-34a, and miR-296, miR-200c, miR-205, miR-92a, miR-20a, miR-378a, miR-19b, miR-17, miR-183, miR-92b, miR-181b, miR-19a, miR-18a, miR-708, miR-92a-1, miR-584, miR-514a, miR-944, and miR-205, miR-152, miR-455, miR-218, miR-143, miR-889, miR-138, miR-382, miR-199a, miR-487b, miR-134, miR-199a, miR-369, miR-494, miR-381, miR-10b, miR-145, miR-410, miR-199b, miR-329, miR-654, miR-376c, miR-409, miR-199b, miR-758, miR-369, miR-495, miR-145, miR-379, miR-323a, miR-377, miR-411, miR-487a, miR-539, miR-323b, miR-380, miR-412, miR-655, miR-1185-1, miR-127, miR-337, miR-382, miR-485, miR-654, miR-143, miR-370, miR-376a, miR-377, miR-432, miR-485, miR-543, miR-10b, miR-1185-2, miR-136, miR-136, miR-154, miR-154, miR-214, miR-214, miR-299, miR-299, miR-337, miR-431, miR-433, miR-490, miR-490, miR-493, miR-493, miR-539, miR-656, miR-665, and combinations thereof (Combination 16).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-125a, miR-99b, miR-182, miR-93, miR-148b, miR-425, miR-30d, miR-26b, miR-484, miR-96, miR-185, miR-25, miR-203a, miR-454, miR-7, miR-23b, miR-342, miR-421, miR-106b, miR-141, miR-95, miR-345, miR-429, miR-542, miR-200b, miR-200a, miR-489, miR-618, miR-653, and combinations thereof (Combination 17).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-221, miR-100, miR-22, miR-29a, miR-320a, miR-222, miR-31, miR-30c, miR-135b, miR-362, miR-146a, miR-221, miR-10a, miR-30a, miR-30a, miR-486, miR-582, miR-196a, miR-1271, miR-379, miR-409, miR-411, and combinations thereof (Combination 18).

In some embodiments, the vector comprises a first DNA sequence comprising a transgene and a second DNA sequence comprising a MBD, wherein the MBD comprises one or more MBSs, wherein the one or more MBSs are specific for one or more microRNAs selected from the group consisting of: miR-155, let-7i, miR-27b, miR-191, miR-27a, miR-99a, miR-151a, miR-450b, miR-450a, and combinations thereof (Combination 19).

### Compositions

The present disclosure also provides pharmaceutical compositions and diagnostic compositions.

An exemplary pharmaceutical composition comprises any vector according to the disclosure and one or more pharmaceutically acceptable excipients.

### Kits

The present disclosure also provides treatment kits and diagnostic kits.

An exemplary treatment kit of the disclosure can comprise any one of the miRNA-regulated vectors of the disclosure or pharmaceutical compositions comprising any one of the miRNA-regulated vectors of the disclosure.

An exemplary diagnostic kit can comprise any miRNA-regulated vector according to the disclosure and suitable assay reagents, where the kit is used to diagnose breast cancer, useful for any stage of breast cancer, including an early stage breast cancer, or a late stage breast cancer. The kit can be configured for in vitro, ex vivo, or in vivo use.

Kits generally further comprise instructions for use.

### Treatment Methods

The present disclosure also provides methods for treating breast cancer. The breast cancer treated according to the methods of the disclosure include an early stage breast cancer or a late stage breast cancer. The guidelines for various stages of breast cancer can be found at the AJCC Cancer Staging Manual, 7th Edition.

As provided herein, a method for treating breast cancer comprises administering a therapeutically effective amount of any one of the vectors of the present disclosure to a subject in need thereof. In some embodiments, the method comprises administering a vector in combination with a second therapeutic agent such as gancyclovir. As referred to herein, as subject can be any mammal, e.g. a humans, a money (e.g. a cynomologous money), companion animals (e.g. cats, dogs) etc.

Various routes of administration can be used, e.g. parenteral (e.g. intravenous, intramuscular, subcutaneous, etc.), oral, nasal (e.g. nebulizer, inhaler, etc.), transmucosal (buccal, nasal mucosal, etc.), and transdermal.

In some embodiments, the present disclosure allows for the development of patient-specific therapeutic compositions and methods. For example, a vector can be designed based on a patient's specific genetic profile, e.g., by screening a sample obtained from the patient, to determine the microRNAs down-regulated in specific target cells (e.g., breast cancer cells) compared to non-target cells. The vector will then include a MBD that comprises one or more MBSs specific for the microRNAs down-regulated or absent in that particular patient's target cells (e.g., breast cancer cells) but not down-regulated in the patient's non-target cells (e.g. non-breast cancer cells). Such a vector can be considered to be a personalized therapeutic agent that can then be delivered to the patient to facilitate the expression of a transgene specifically in target cells (e.g., late stage breast cancer cells) while providing minimal or no expression of the transgene in non-target cells.

In some embodiments, to obtain a patient's genetic profile (e.g. a miRNA profile), biopsies from previously diagnosed or undiagnosed tissue samples can be obtained. If the tissue is undiagnosed, diagnosis can be achieved using standard pathological methods in-house. Once diagnosed, the biopsied tissue can be processed in many ways. For example, in some embodiments the biopsied tissue can be sectioned. In such embodiments, one portion of the biopsy can be stored in long term storage (i.e. frozen at −80° C. or stored in liquid nitrogen), one portion can be put into cell culture, and one portion can be analyzed for its genetic profile. This genetic profile can be confirmed and compared against biopsies of surrounding, healthy tissue, and tissue from other major and accessible tissue in the body, which may also include the vital organs of the patient.

Genetic Analysis—Establishing “miRNA Signature” from Biopsies

In some embodiments, a biopsied tissue can be profiled for specific biomarkers, including total miRNA sequences. A “miRNA Signature” for the tissue type can be generated from data gathered and used as an identifier to differentiate between cell types. Direct comparisons may be made between cell types (e.g. healthy cells and diseased cells, different tissue types, etc.) via their miRNA signatures. This comparison comprises finding differences in relative levels of expression of miRNAs. Relative levels of expression of miRNAs indicate how particular miRNAs are down/up-regulated between cell types, after being normalized to a standard. For example: given 3 miRNAs: miRNA1, miRNA2, and miRNA3, in two cell types: Cell1 and Cell2, the following may occur. In Cell1, miRNA1 may be expressed at a normalized value of 1.0, miRNA2 may not be expressed, and miRNA 3 may be expressed at a normalized value of 1.5. In Cell2, miRNA1 may not be expressed, miRNA2 may be expressed at a normalized value of 1.0, and miRNA3 may also be expressed at a normalized value of 1.5. In this case, the RNA expression between Cell1 and Cell2 indicate that miRNA1 and miRNA2 have different levels relative to each other, while miRNA3 have equal levels of expression relative to each other. This means that miRNA1 and miRNA2 could be used as potential targets to allow for cell-type specific targeting between the Cell1 and Cell2. This technique can be scaled up to obtain a complete miRNA signatures for various cell types. The miRNA signatures can be obtained from patient data of different organs and a “general miRNA signature” can be established for each organ. Patient-specific, tissue-specific signatures can be established as well. These patient-specific miRNA signatures would allow development of a patient-specific therapeutic vector according to the disclosure.

### Diagnostic Methods

The present disclosure provides methods for diagnosing breast cancer. In various embodiments, a diagnosis can be performed in vivo, in vitro, or ex vivo.

In some embodiments, a method for diagnosing a breast cancer comprises (a) introducing a vector of the present disclosure comprising a reporter transgene into a breast tissue; (b) measuring the expression of the reporter transgene; (c) comparing the expression of the reporter transgene to a control; and (d) diagnosing the subject as having breast cancer or not having breast cancer.

A vector comprising a reporter transgene may be introduced into a breast tissue in vivo or ex vivo. Alternatively, a breast biopsy sample may be obtained from a patient and the vector comprising a reporter transgene may be introduced into the breast biopsy sample in vitro.

In some embodiments, a control can be a biopsy sample transfected with a control vector where the MBSs are replaced by flipped sequences (i.e. the sequence of the MBS reversed). In other embodiments, the control can be a normal breast cell transfected with the vector comprising MBSs according to the present disclosure. In yet other embodiments, the control can be a non-breast cell treated with a vector of the present disclosure. In some embodiments, the vectors of the present disclosure could be used for diagnosing various stages of breast cancer.

It is to be understood that the terminology employed herein is used for the purpose of describing particular embodiments only and is not intended to be limiting since the scope of the present disclosure will be limited only by the appended claims and equivalents thereof. The following examples are for illustrative purposes. These are intended to show certain aspects and embodiments of the present disclosure but are not intended to limit the disclosure in any manner.

### EXAMPLES

### Example 1: Identification of miRNAs Down-Regulated in Breast Cancer Cells Compared to Normal Breast Cells

MicroRNA sequencing data for three cell lines, MCF7 (early stage breast cancer cell line), MCF10A (normal breast cell line), and BT549 (late stage breast cancer cell line), was generated. Cell lines MCF7, MCF10A, and BT549 were shipped to a service provider for sequencing. Sequencing data was generated through the Next-Generation Sequencing and returned to the inventor for analysis. The data was analyzed and miRNAs differentially expressed in these cell lines were identified. Table 6 lists exemplary miRNAs differentially expressed in these cell lines.

TABLE 6

| MicroRNAs down- | MicroRNAs down- | | |
|---|---|---|---|
| regulated in late | regulated in early | MicroRNAs down- | MicroRNAs down- |
| stage breast cancer | stage breast cancer | regulated in early | regulated in late |
| cells, not down- | cells, not down- | stage breast cancer | stage breast cancer |
| regulated in early | regulated in late | cells, not down- | cells, not down- |
| stage breast cancer | stage breast cancer | regulated in normal | regulated in normal |
| cells | cells | breast cells | cells |
| 629 | 452 | 224 | 205 |
| 200C | 224 | 577 | 200C |
| 203A | 100 | 452 | 510 |
| 4760 | 31 | 221 | |
| 429 | 10A | 100 | |
| 95 | | 205 | |
| 489 | | 31 | |

The sequencing data showed that certain miRNAs, e.g., miR-205-5p and miR-34c-5p, were expressed at significantly low levels or were completely silenced in both MCF7 and BT549. Cell-type specific miRNAs, such as miR-203c-3p, exhibited significant downregulation only in BT549.

### Example 2: Generation of miRNA-Regulated GFP Constructs and Analysis of GFP Expression in Breast Cancer Cells and Normal Breast Cells

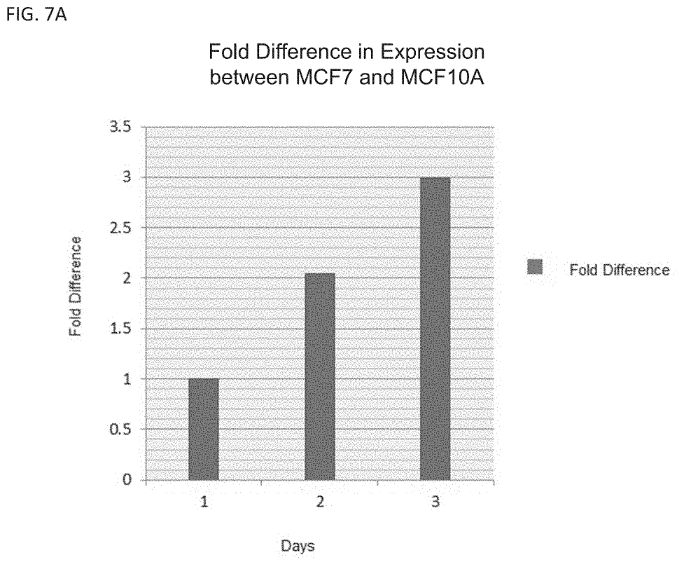

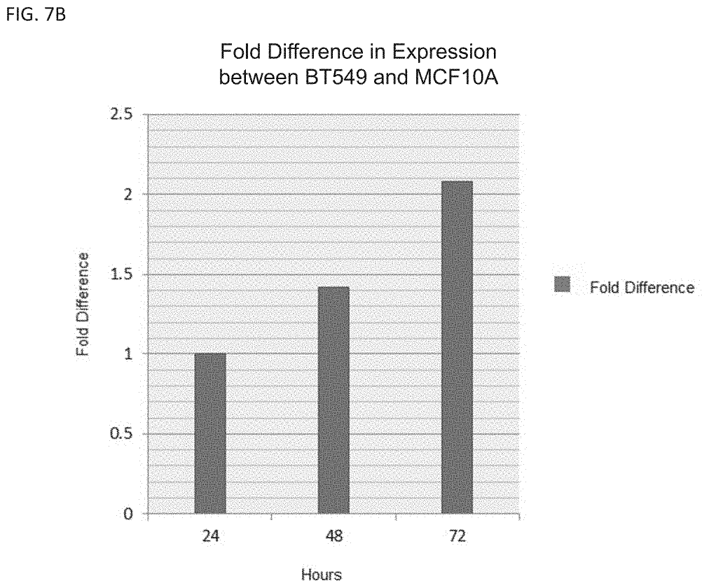

pSELECT-zeo-HSV1tk plasmid vector was obtained from Invivogen (FIG. 7). This plasmid contains a non-CpG, codon optimized Herpes-Simplex Virus Thymidine Kinase (HSV-TK) analog downstream of a conjugated hEF1-HTLV promoter. HSV-TK is an apoptosis inducer that metabolizes a prodrug, Gancyclovir, into a toxic substance that inhibits DNA synthesis in the cell. The inventors replaced HSV1-TK with Green Fluorescent Protein in order to turn it into a reporter system.

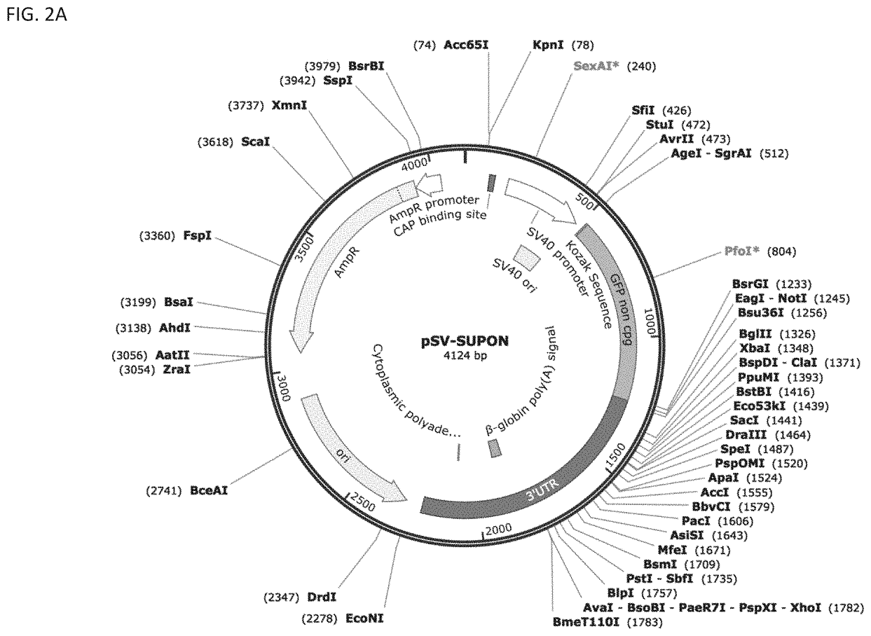

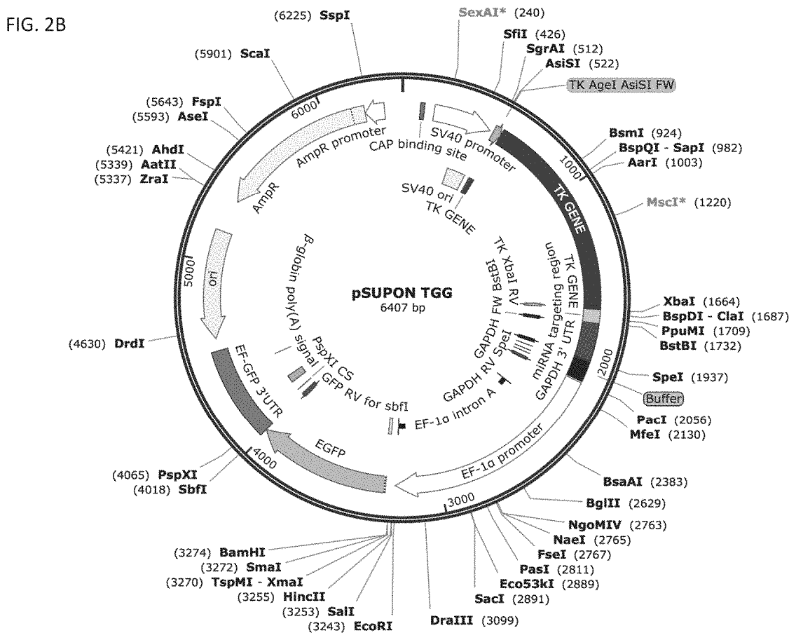

To generate an miRNA-regulated transgene vector, (test construct/vector/plasmid), sequences complementary to 5 miRNAs, miR-100, miR-138, miR-221, miR-222, and miR-205, were incorporated as MBSs in the plasmid vector. Three copies of each MBS were inserted into the 3′ UTR of a GFP-expressing construct with 15-17 nucleotide spacer sequences between each copy of the MBSs. A plasmid containing the flipped (untargeted) sequences in place of MBSs was generated as a control. Additionally, the 3′ UTR of the GAPDH gene was inserted downstream (3′) of MBSs in the test and control plasmids. An exemplary miRNA-regulated transgene vector is shown in FIG. 2B.

MCF7 and BT549 cells were cultured in DMEM high glucose with supplemented glutamine, 10% FBS, and 1× pen-strep. MCF10A cells were cultured in DMEM:F/12 medium containing 5% Horse Serum, 20 ng/ml EGF, 0.5 mg/ml Hydrocortisone, 100 ng/ml Cholera Toxin, 10 μg/ml Insulin, with 1× pen strep.

Cells were transfected with both flipped (control vector) and test GFP constructs (miRNA regulated vector) using Lipofectamine 3000 from Thermo Fisher.

Transfected cells were analyzed via image analysis and quantification using Keyence BX 710 series fluorescence microscope and software. The centers of the wells were defined and pictures were taken in 3×3 grids around the centers, i.e., 9 pictures were taken in each well. Exposures and aperture settings remained consistent between samples. Pictures were stitched together using Keyence software, and cells were counted using “Hybrid cell count” feature. To determine transfection efficiencies, total cell counts were taken in both Bright Field pictures as well as corresponding GFP pictures. GFP cell numbers were then divided by bright field cell numbers and multiplied by 100, yielding the expression percentage per well in a given cell line.

The expression percentage was then normalized to the control construct (expression percentage of control construct divided by expression percentage of test construct). This yielded the fold difference of expression between control and test constructs of a given cell line or Fd. The Fd of respective cell lines was divided to determine the normalized fold difference between cell lines. This approach allowed determining the fold difference in regulation between MCF7 and MCF10A using identical constructs.

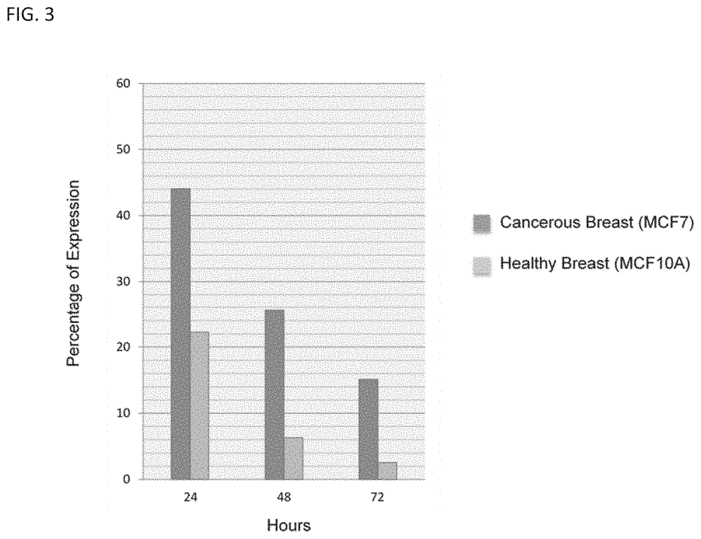

FIG. 3 shows that the miRNA-regulated GFP transgene is expressed in early stage breast cancer cells (MCF7) whereas the GFP expression in healthy breast cells (MCF10A) using the same construct is minimal.

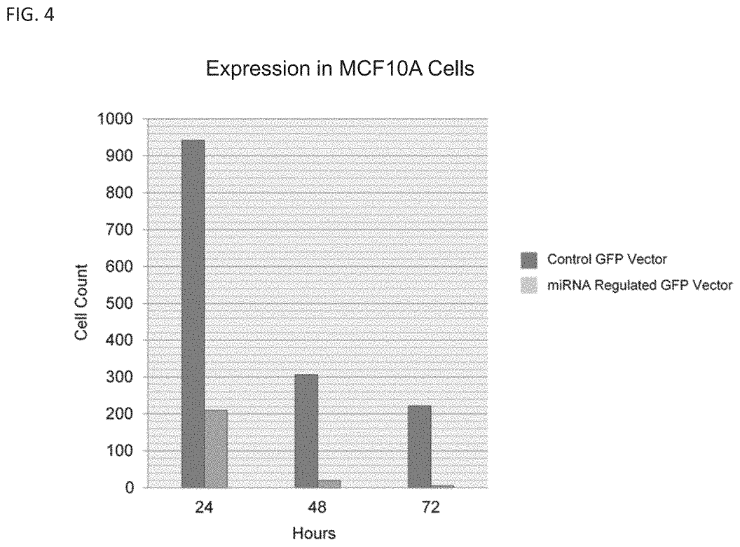

FIG. 4 demonstrates that healthy breast cells (MCF10A) transfected with the control GFP vector, where the GFP transgene is not regulated by miRNAs, show the GFP expression whereas MCF10A cells transfected with the miRNA-regulated GFP expression vector show a minimal expression of GFP.

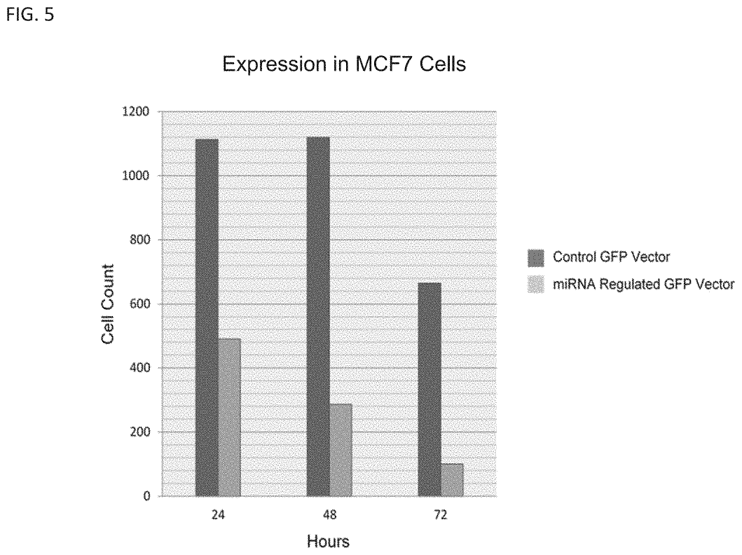

FIG. 5 demonstrates that early stage breast cancer cells (MCF7) transfected with the control GFP vector show the GFP expression and MCF7 cells transfected with the miRNA-regulated GFP expression vector also show the GFP expression.

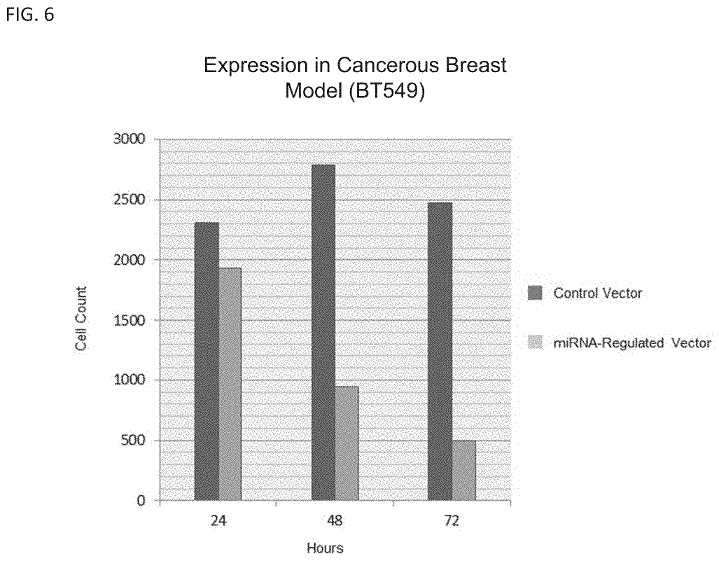

FIG. 6 demonstrates that triple negative breast cancer cells (BT549) transfected with the control vector show the GFP expression and BT549 cells transfected with the miRNA-regulated expression vector also show the GFP expression. Compared to MCF7 cells (FIG. 5), the expression differential between the control vector and the miRNA-regulated vector is lower in BT549 cells because the level of expression of miRNAs specific for the MBSs present in the vector is lower in BT549 compared to MCF7.

The GFP expression data for MCF7 cells and MCF10A cells was normalized to account for both the transfection efficiency and standard expression decay over time. The normalized fold difference of GFP expression over time between healthy breast cells, MCF10A, and early stage breast cancer cells, MCF7, is shown in FIG. 7A. These data show that the miRNA-regulated vector expressed about 1-3 fold stronger in MCF7 cells compared to MCF10A cells. The normalized fold difference of GFP expression over time between healthy breast cells, MCF10A, and triple negative breast cancer cells, BT549, is shown in FIG. 7B.

### Example 3: Generation of miRNA-Regulated Plasmid Construct that Induces Apoptosis in Triple Negative Breast Cancer Cells but not in Healthy Breast Cells when Expressed in Conjunction with a Prodrug

pSUPON plasmid DNA sequence was designed using the Snapgene software. pSUPON encodes for a non-CpG GFP analog controlled by an SV-ori promoter and enhancer. 3-prime to the GFP sequence lies many unique restriction enzyme sites for cloning of miRNA regulatory sites downstream of the open reading frame, allowing for the customization of the expressed gene. This plasmid is designed to function under methylated conditions, which has been implicated in published literature to mitigate foreign DNA catalyzed immunogenicity.

This pSUPON plasmid was modified to contain the HSV1tk gene from the pSELECT vector in place of the non-CpG GFP. Additionally, a separate Open Reading Frame was added that contained the eGFP sequence 3′ to a new EF1a promoter. This allowed for the vector to express both HSV1tk and GFP concurrently, allowing identification of the cells successfully expressing the vector.

5′ to this new Open Reading Frame, the 3′UTR of GAPDH was inserted to serve as the terminating sequence and regulatory element for the HSV1tk gene. Collectively, this vector is called pSUPON-TGG (“TGG” represents TK, GAPDH, and GFP).

In order to make this vector regulated by miRNA sequences, four complementary target sequences for microRNA 205-5p were inserted between the stop codon for HSV1tk and the GAPDH sequence in the UTR. microRNA205-5p was shown to have high expression in MCF10A cells (healthy breast) but very low expression in BT549 cells (triple negative breast cancer) cells. The EF1α-GFP motif was not regulated by the miRNA 205-5p target sequences. This allowed for the regulation of only the TK gene, and not the GFP.

The vectors were transfected twice at 24 hours intervals (once at 0 hours and again at 24 hours) into both MCF10A cells and BT549 cells using Lipofectamine 3000 from Thermo Fisher. Four wells total were transfected: two MCF10A wells, and two BT549 wells of a 12 well plate.

48 hours post-transfection, one transfected well from each cell line was treated with media containing 10 μm Gancyclovir for a period of 10 days. One well remained untreated to be used as a negative control (untreated). The media was changed every day.

Cells were imaged each day, 24 hours apart, after feeding. GFP-expressing cells were counted using the Keyence Analyzer software as described in Example 2, except transfection efficiencies were not obtained. Raw cell count was measured and compared between wells. The Keyence machine was not able to capture the full well, and so the area of the captured portion of the well was measured using photoshop, and the cell number was adjusted to reflect the total area of the well (i.e. roughly 80% of the treated well was captured, so the cell number was multiplied by roughly 1.2 to be normalized to the fully captured untreated well).

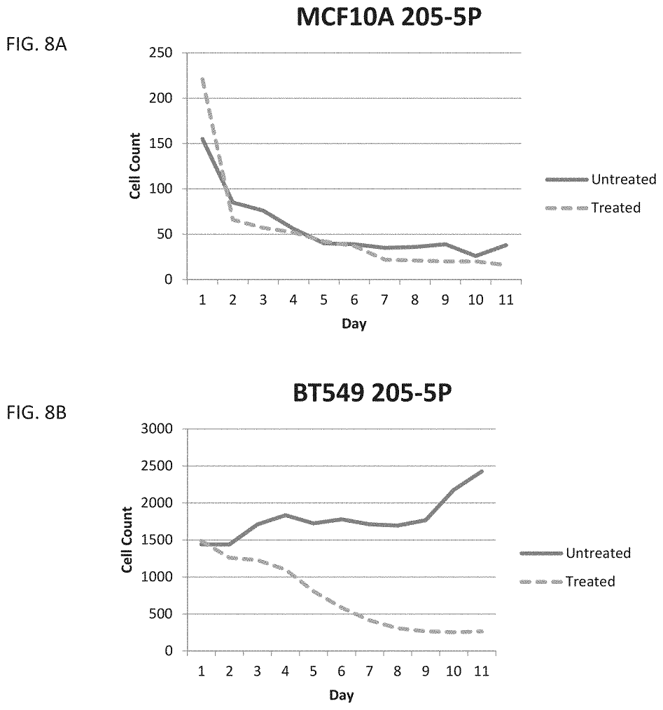

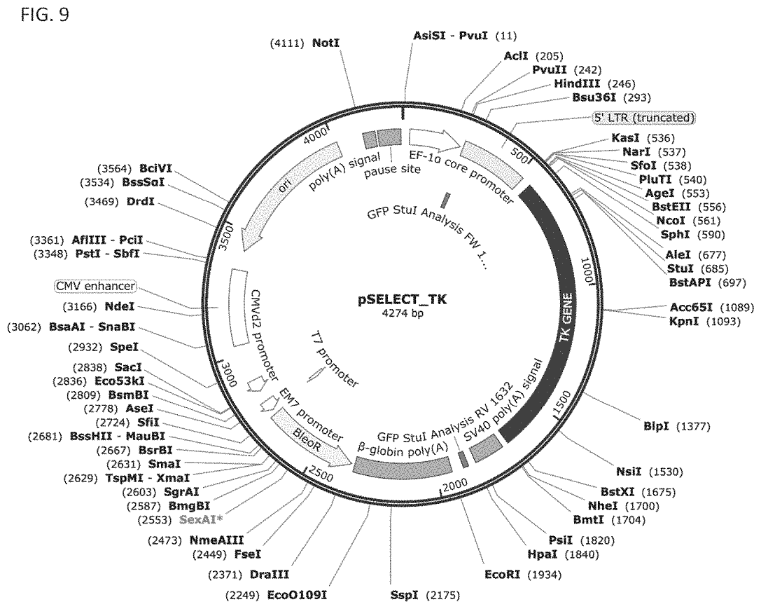

FIG. 8A reflects the total cell count over time (normalized to reflect a whole well) of GFP expressing cells in both the Gancyclovir treated and untreated MCF10A cells. Expression decreased over time; however, the data clearly indicated that the miRNA-205-5p target sites were facilitating the downregulation of the TK gene in the MCF10A cells where miRNA-205 is present in abundance.

FIG. 8B reflects the total cell count over time (normalized to reflect a whole well) of GFP expressing cells in both the Gancyclovir treated and untreated BT549 cells. Expression of the untreated cells (solid) increased over time while the treated cells (dashed) were significantly reduced in number indicating that the miRNA205-5p target sites did not affect the expression and activity of TK in the BT549 cells where miRNA-205 is absent.

### Example 4: Next Generation Sequencing (NGS) of Total miRNA Profiles of Breast Cancer Cells and Healthy Cells

NGS was used to measure total miRNA profiles of MCF10A (healthy breast cells), BT549 (triple negative breast cancer cells), MCF7 (early stage breast cancer cells), and CCD1070sk (healthy skin fibroblast) cell lines. Raw data for the total read count for selected miRNAs is shown in Tables 7-12. The higher the number, the greater the expression. miRNAs were selected based on their differential expression.

Table 7 shows miRNAs upregulated or expressed abundantly in healthy cells (CCD1070sk or MCF10A) but down-regulated in cancer cells (BT549 or MCF7). The vectors of the present disclosure can comprise MBSs for these miRNAs to regulate the expression of the transgene in breast cancer cells.

TABLE 7

| miRNA | CCD10705.1 | BT549.1 | MCF7.1 | MCF10a.1 | CCD10705.2 | BT549.2 | MCF7.2 | MCF10a.2 |
|---|---|---|---|---|---|---|---|---|
| hsa-let-7b-5p | 91515 | 34895 | 18610 | 96087 | 68830 | 54671 | 30590 | 120215 |
| hsa-miR-423-3p | 66651 | 26488 | 36695 | 91147 | 53138 | 38636 | 59572 | 96191 |
| hsa-miR-423-5p | 20152 | 16105 | 6209 | 29240 | 15081 | 17076 | 8755 | 30132 |
| hsa-miR-34c-5p | 20658 | 505 | 37 | 15287 | 15482 | 565 | 40 | 14822 |
| hsa-miR-34a-5p | 8346 | 330 | 5310 | 6032 | 6860 | 331 | 7146 | 7006 |
| hsa-miR-296-3p | 424 | 1 | 1021 | 242 | 394 | 5 | 1315 | 310 |

Table 8 shows miRNAs upregulated or expressed abundantly in healthy breast cells (MCF10A) but down-regulated in cancer cells (BT549 or MCF7). The vectors of the present disclosure can comprise MBSs for these miRNAs to regulate the expression of the transgene in breast cancer cells.

TABLE 8

| miRNA | CCD10705.1 | BT549.1 | MCF7.1 | MCF10a.1 | CCD10705.2 | BT549.2 | MCF7.2 | MCF10a.2 |
|---|---|---|---|---|---|---|---|---|
| hsa-miR-200c-3p | 61 | 119 | 213615 | 203862 | 61 | 108 | 288421 | 213098 |
| hsa-miR-205-5p | 13 | 8 | 242 | 184051 | 31 | 5 | 335 | 183554 |
| hsa-miR-92a-3p | 17641 | 58978 | 29025 | 175281 | 13781 | 65312 | 39938 | 202828 |
| hsa-miR-20a-5p | 9476 | 29578 | 11102 | 111530 | 6292 | 29348 | 15753 | 111768 |
| hsa-miR-378a-3p | 3288 | 22738 | 16162 | 83193 | 2896 | 24283 | 20215 | 97853 |
| hsa-miR-19b-3p | 4180 | 9969 | 3271 | 30767 | 2875 | 8279 | 4151 | 25118 |
| hsa-miR-17-5p | 2549 | 7818 | 2818 | 29259 | 1946 | 8111 | 4331 | 29482 |
| hsa-miR-183-5p | 58 | 17213 | 63106 | 23355 | 46 | 14933 | 75454 | 21516 |
| hsa-miR-92b-3p | 9892 | 16602 | 9893 | 22128 | 8586 | 23355 | 13824 | 29222 |
| hsa-miR-181b-5p | 4816 | 4008 | 9250 | 12717 | 3800 | 4623 | 12063 | 13516 |
| hsa-miR-19a-3p | 1277 | 3061 | 956 | 7145 | 881 | 2302 | 1273 | 5880 |
| hsa-miR-18a-5p | 747 | 2271 | 797 | 7085 | 540 | 2039 | 1037 | 6718 |
| hsa-miR-708-5p | 1409 | 31 | 376 | 6570 | 1215 | 15 | 490 | 5875 |
| hsa-miR-92a-1-5p | 280 | 1567 | 789 | 6061 | 198 | 1533 | 814 | 6067 |
| hsa-miR-584-5p | 521 | 194 | 269 | 4326 | 375 | 238 | 341 | 4962 |
| hsa-miR-514a-3p | 1 | 2 | 1 | 312 | 0 | 1 | 1 | 260 |
| hsa-miR-944 | 0 | 2 | 5 | 252 | 0 | 2 | 5 | 217 |
| hsa-miR-205-3p | 0 | 0 | 3 | 212 | 0 | 0 | 1 | 211 |

Table 9 shows miRNAs upregulated or expressed abundantly in healthy human primary fibroblasts (CCD1070sk) but down-regulated in cancer cells (BT549 or MCF7). The vectors of the present disclosure can comprise MBSs for these miRNAs to regulate the expression of the transgene in breast cancer cells.

TABLE 9

| miRNA | CCD10705.1 | BT549.1 | MCF7.1 | MCF10a.1 | CCD10705.2 | BT549.2 | MCF7.2 | MCF10a.2 |
|---|---|---|---|---|---|---|---|---|
| hsa-miR-152-3p | 48335 | 8840 | 8495 | 777 | 43542 | 7307 | 10350 | 768 |
| hsa-miR-455-3p | 13934 | 291 | 595 | 614 | 12244 | 256 | 692 | 634 |
| hsa-miR-218-5p | 6668 | 3519 | 410 | 204 | 5898 | 2074 | 374 | 188 |
| hsa-miR-143-3p | 236313 | 78 | 312 | 83 | 223305 | 51 | 446 | 70 |
| hsa-miR-889-3p | 9981 | 1724 | 4 | 68 | 8383 | 1026 | 10 | 58 |
| hsa-miR-138-5p | 1039 | 91 | 3 | 55 | 870 | 106 | 5 | 55 |
| hsa-miR-382-5p | 5867 | 1329 | 0 | 26 | 4690 | 1032 | 7 | 33 |
| hsa-miR-199a-5p | 331917 | 51 | 69 | 24 | 273117 | 24 | 225 | 16 |
| hsa-miR-487b-3p | 4426 | 740 | 3 | 24 | 3409 | 631 | 6 | 28 |
| hsa-miR-134-5p | 4716 | 662 | 0 | 23 | 4273 | 518 | 2 | 29 |
| hsa-miR-199a-3p | 260252 | 56 | 153 | 23 | 200950 | 36 | 275 | 6 |
| hsa-miR-369-3p | 5790 | 891 | 11 | 21 | 4178 | 428 | 10 | 15 |
| hsa-miR-494-3p | 1851 | 478 | 11 | 20 | 1540 | 387 | 3 | 22 |
| hsa-miR-381-3p | 9543 | 916 | 1 | 19 | 8937 | 569 | 5 | 11 |
| hsa-miR-10b-5p | 21838 | 284 | 71 | 17 | 16055 | 212 | 55 | 14 |
| hsa-miR-145-5p | 59237 | 6 | 14 | 16 | 50490 | 9 | 52 | 40 |
| hsa-miR-410-3p | 1380 | 137 | 1 | 13 | 1044 | 101 | 1 | 5 |
| hsa-miR-199b-3p | 129218 | 27 | 74 | 12 | 100932 | 17 | 123 | 6 |
| hsa-miR-329-3p | 2814 | 362 | 0 | 12 | 2195 | 335 | 6 | 10 |
| hsa-miR-654-3p | 6898 | 647 | 0 | 11 | 6349 | 426 | 4 | 12 |
| hsa-miR-376c-3p | 2897 | 325 | 2 | 10 | 2297 | 225 | 2 | 6 |
| hsa-miR-409-3p | 8099 | 823 | 1 | 10 | 7109 | 845 | 3 | 19 |
| hsa-miR-199b-5p | 143069 | 33 | 170 | 9 | 107220 | 32 | 260 | 13 |
| hsa-miR-758-3p | 2597 | 285 | 1 | 8 | 2186 | 237 | 1 | 2 |
| hsa-miR-369-5p | 2743 | 255 | 0 | 6 | 2276 | 232 | 2 | 11 |
| hsa-miR-495-3p | 559 | 209 | 0 | 6 | 465 | 174 | 2 | 5 |
| hsa-miR-145-3p | 6832 | 2 | 4 | 5 | 5444 | 0 | 7 | 3 |
| hsa-miR-379-3p | 2085 | 336 | 0 | 5 | 1538 | 242 | 1 | 6 |
| hsa-miR-323a-3p | 489 | 82 | 1 | 4 | 431 | 81 | 0 | 6 |
| hsa-miR-377-3p | 698 | 99 | 0 | 4 | 488 | 56 | 0 | 2 |
| hsa-miR-411-3p | 4358 | 669 | 2 | 4 | 3353 | 509 | 10 | 8 |
| hsa-miR-487a-3p | 866 | 152 | 0 | 4 | 694 | 134 | 0 | 3 |
| hsa-miR-539-5p | 217 | 58 | 0 | 4 | 189 | 50 | 0 | 2 |
| hsa-miR-323b-3p | 308 | 60 | 0 | 3 | 262 | 61 | 0 | 3 |
| hsa-miR-380-3p | 432 | 91 | 1 | 3 | 377 | 35 | 1 | 3 |
| hsa-miR-412-5p | 518 | 130 | 3 | 3 | 374 | 99 | 0 | 1 |
| hsa-miR-655-3p | 924 | 178 | 0 | 3 | 596 | 111 | 0 | 0 |
| hsa-miR-1185-1-3p | 840 | 180 | 1 | 2 | 697 | 136 | 1 | 2 |
| hsa-miR-127-3p | 63027 | 6189 | 20 | 2 | 60895 | 6371 | 59 | 3 |
| hsa-miR-337-5p | 2059 | 223 | 0 | 2 | 2115 | 208 | 1 | 0 |
| hsa-miR-382-3p | 315 | 105 | 0 | 2 | 247 | 46 | 1 | 0 |
| hsa-miR-485-3p | 517 | 65 | 0 | 2 | 428 | 126 | 1 | 1 |
| hsa-miR-654-5p | 1271 | 176 | 1 | 2 | 989 | 159 | 0 | 2 |
| hsa-miR-143-5p | 696 | 0 | 0 | 1 | 675 | 0 | 0 | 1 |
| hsa-miR-370-3p | 22648 | 1572 | 5 | 1 | 21312 | 1742 | 20 | 0 |
| hsa-miR-376a-3p | 1428 | 184 | 1 | 1 | 1068 | 124 | 2 | 0 |
| hsa-miR-377-5p | 1158 | 110 | 0 | 1 | 961 | 106 | 1 | 3 |
| hsa-miR-432-5p | 4316 | 590 | 1 | 1 | 3282 | 495 | 5 | 0 |
| hsa-miR-485-5p | 619 | 47 | 0 | 1 | 501 | 68 | 1 | 4 |
| hsa-miR-543 | 841 | 185 | 1 | 1 | 609 | 195 | 0 | 6 |
| hsa-miR-10b-3p | 398 | 5 | 0 | 0 | 250 | 4 | 0 | 0 |
| hsa-miR-1185-2-3p | 645 | 148 | 0 | 0 | 541 | 98 | 1 | 0 |
| hsa-miR-136-3p | 1250 | 245 | 0 | 0 | 1062 | 126 | 2 | 0 |
| hsa-miR-136-5p | 225 | 51 | 0 | 0 | 195 | 20 | 1 | 0 |
| hsa-miR-154-3p | 485 | 82 | 0 | 0 | 319 | 65 | 1 | 0 |
| hsa-miR-154-5p | 109 | 13 | 0 | 0 | 86 | 13 | 0 | 1 |
| hsa-miR-214-3p | 12065 | 1 | 3 | 0 | 11620 | 2 | 10 | 0 |
| hsa-miR-214-5p | 170 | 0 | 0 | 0 | 149 | 0 | 1 | 0 |
| hsa-miR-299-3p | 203 | 61 | 0 | 0 | 175 | 46 | 1 | 0 |
| hsa-miR-299-5p | 167 | 33 | 0 | 0 | 157 | 80 | 0 | 0 |
| hsa-miR-337-3p | 1961 | 235 | 0 | 0 | 1544 | 182 | 1 | 0 |
| hsa-miR-431-5p | 808 | 418 | 0 | 0 | 740 | 376 | 0 | 0 |
| hsa-miR-433-3p | 1687 | 207 | 2 | 0 | 1477 | 285 | 3 | 0 |
| hsa-miR-490-3p | 131 | 1 | 0 | 0 | 121 | 0 | 1 | 0 |
| hsa-miR-490-5p | 349 | 3 | 0 | 0 | 447 | 1 | 0 | 0 |
| hsa-miR-493-3p | 4966 | 535 | 0 | 0 | 4153 | 393 | 6 | 0 |
| hsa-miR-493-5p | 13122 | 1388 | 1 | 0 | 9413 | 1001 | 11 | 0 |
| hsa-miR-539-3p | 256 | 61 | 0 | 0 | 206 | 23 | 0 | 1 |
| hsa-miR-656-3p | 1189 | 104 | 0 | 0 | 1084 | 42 | 0 | 0 |
| hsa-miR-665 | 306 | 124 | 0 | 0 | 519 | 108 | 0 | 0 |

Table 10 shows miRNAs upregulated in breast cancer cells (BT549 or MCF7) but down-regulated in healthy cells (CCD1070sk or MCF10A). The vectors of the present disclosure can comprise MBSs for these miRNAs to regulate the expression of the transgene in healthy cells.

TABLE 10

| miRNA | CCD10705.1 | BT549.1 | MCF7.1 | MCF10a.1 | CCD10705.2 | BT549.2 | MCF7.2 | MCF10a.2 |
|---|---|---|---|---|---|---|---|---|
| hsa-miR-155-5p | 31583 | 26775 | 145 | 3 | 23008 | 25364 | 195 | 2 |
| hsa-let-7i-5p | 335652 | 547751 | 331935 | 168567 | 247626 | 467621 | 407513 | 162614 |
| hsa-miR-27b-3p | 141326 | 234228 | 143960 | 88751 | 124939 | 162033 | 146032 | 89283 |
| hsa-miR-191-5p | 54690 | 122198 | 244019 | 76512 | 46151 | 106631 | 277649 | 76628 |
| hsa-miR-27a-3p | 59323 | 151700 | 160327 | 73034 | 62920 | 110064 | 150000 | 69622 |
| hsa-miR-99a-5p | 84650 | 96449 | 84823 | 11614 | 73671 | 84887 | 103910 | 9723 |
| hsa-miR-151a-5p | 3491 | 9085 | 7587 | 4415 | 3365 | 7301 | 9152 | 4478 |
| hsa-miR-450b-5p | 4193 | 4614 | 6988 | 282 | 3106 | 2033 | 6254 | 228 |
| hsa-miR-450a-5p | 2347 | 2597 | 4564 | 159 | 1825 | 1297 | 4100 | 138 |

Table 11 shows miRNAs upregulated in early stage breast cancer cells (MCF7) but down-regulated in healthy cells (CCD1070sk or MCF10A) or late stage breast cancer cells (BT549). The vectors of the present disclosure can comprise MBSs for these miRNAs to regulate the expression of the transgene in late stage breast cancer cells or healthy breast cells.

TABLE 11

| miRNA | CCD10705.1 | BT549.1 | MCF7.1 | MCF10a.1 | CCD10705.2 | BT549.2 | MCF7.2 | MCF10a.2 |
|---|---|---|---|---|---|---|---|---|
| hsa-miR-125a-5p | 169094 | 94447 | 209382 | 275714 | 147030 | 101799 | 286557 | 300882 |
| hsa-miR-99b-5p | 124234 | 76296 | 214259 | 148773 | 105564 | 101548 | 308790 | 146151 |
| hsa-miR-182-5p | 266 | 79834 | 196322 | 95656 | 224 | 75636 | 247215 | 84920 |
| hsa-miR-93-5p | 17726 | 40654 | 86539 | 64418 | 12205 | 44671 | 121008 | 68219 |
| hsa-miR-148b-3p | 30891 | 41085 | 59060 | 30267 | 26034 | 28451 | 67334 | 27721 |
| hsa-miR-425-5p | 9633 | 31906 | 132610 | 27909 | 7963 | 32933 | 188036 | 29158 |
| hsa-miR-30d-5p | 3055 | 24428 | 38898 | 12414 | 2735 | 26334 | 52359 | 12766 |
| hsa-miR-26b-5p | 7440 | 9982 | 33317 | 11785 | 5832 | 4660 | 30592 | 8699 |
| hsa-miR-484 | 16871 | 15238 | 23979 | 11079 | 15236 | 12778 | 27373 | 10486 |
| hsa-miR-96-5p | 30 | 13446 | 41162 | 8388 | 33 | 8627 | 42706 | 6226 |
| hsa-miR-185-5p | 11875 | 8890 | 17441 | 8193 | 9186 | 7576 | 21345 | 8247 |
| hsa-miR-25-3p | 4104 | 9049 | 16726 | 6682 | 3389 | 10999 | 23071 | 7102 |
| hsa-miR-203a-3p | 92 | 84 | 79345 | 6010 | 89 | 29 | 97383 | 6165 |
| hsa-miR-454-3p | 3217 | 5336 | 56859 | 5553 | 2508 | 4531 | 73107 | 4813 |
| hsa-miR-7-5p | 5506 | 22758 | 108173 | 5496 | 3680 | 18259 | 124903 | 5687 |
| hsa-miR-23b-3p | 3324 | 17428 | 12299 | 3978 | 3278 | 17831 | 16445 | 5077 |
| hsa-miR-342-3p | 1226 | 2426 | 65556 | 3940 | 1061 | 2752 | 94748 | 3878 |
| hsa-miR-421 | 2343 | 4185 | 13298 | 3340 | 2089 | 3906 | 14919 | 3473 |
| hsa-miR-106b-3p | 1441 | 5829 | 14634 | 2839 | 1323 | 5282 | 17472 | 3323 |
| hsa-miR-141-3p | 5 | 4 | 17956 | 2747 | 2 | 6 | 18896 | 2258 |
| hsa-miR-95-3p | 32 | 48 | 13029 | 2485 | 23 | 44 | 15957 | 2435 |
| hsa-miR-345-5p | 1081 | 2908 | 12766 | 858 | 1024 | 2750 | 14751 | 956 |
| hsa-miR-429 | 6 | 190 | 40215 | 855 | 11 | 109 | 42377 | 789 |
| hsa-miR-542-3p | 11194 | 7935 | 14566 | 771 | 8303 | 6648 | 17349 | 797 |
| hsa-miR-200b-3p | 8 | 134 | 24467 | 607 | 6 | 123 | 31069 | 506 |
| hsa-miR-200a-3p | 6 | 173 | 35209 | 356 | 3 | 119 | 39720 | 321 |
| hsa-miR-489-3p | 0 | 3 | 2500 | 0 | 1 | 2 | 2910 | 1 |
| hsa-miR-618 | 667 | 19 | 998 | 0 | 507 | 14 | 1207 | 0 |
| hsa-miR-653-3p | 0 | 7 | 11460 | 0 | 0 | 11 | 10529 | 0 |

Table 12 shows miRNAs upregulated in late stage breast cancer cells (BT549) but down-regulated in healthy cells (CCD1070sk or MCF10A) or early stage breast cancer cells (MCF7). The vectors of the present disclosure can comprise MBSs for these miRNAs to regulate the expression of the transgene in early stage breast cancer cells or healthy cells.

TABLE 12

| miRNA | CCD10705.1 | BT549.1 | MCF7.1 | MCF10a.1 | CCD10705.2 | BT549.2 | MCF7.2 | MCF10a.2 |
|---|---|---|---|---|---|---|---|---|
| hsa-miR-221-3p | 1994903 | 563833 | 7086 | 986589 | 1520681 | 485662 | 10182 | 921854 |
| hsa-miR-100-5p | 987893 | 362692 | 521 | 433230 | 797711 | 319851 | 1187 | 355570 |
| hsa-miR-22-3p | 216394 | 112516 | 34560 | 74926 | 181120 | 93098 | 41636 | 72012 |
| hsa-miR-29a-3p | 150411 | 105315 | 7713 | 49169 | 133217 | 83198 | 7738 | 41214 |
| hsa-miR-320a | 19801 | 71561 | 10858 | 46877 | 14549 | 51849 | 13269 | 48946 |
| hsa-miR-222-3p | 21367 | 25610 | 623 | 12934 | 20429 | 18861 | 649 | 13479 |
| hsa-miR-31-5p | 27460 | 33854 | 41 | 6751 | 25903 | 31584 | 84 | 7038 |
| hsa-miR-30c-5p | 4198 | 21346 | 3668 | 6429 | 3469 | 16687 | 4255 | 5795 |
| hsa-miR-135b-5p | 44 | 8262 | 371 | 5719 | 37 | 4295 | 389 | 4392 |
| hsa-miR-362-5p | 3015 | 11263 | 4319 | 5035 | 2340 | 11858 | 5893 | 4766 |
| hsa-miR-146a-5p | 9682 | 47526 | 108 | 1850 | 7715 | 36993 | 103 | 1586 |
| hsa-miR-221-5p | 5109 | 3857 | 79 | 1810 | 3497 | 3519 | 100 | 1530 |
| hsa-miR-10a-5p | 729 | 124166 | 371 | 1768 | 615 | 113201 | 435 | 1611 |
| hsa-miR-30a-5p | 2229 | 23026 | 560 | 1694 | 1841 | 20080 | 671 | 1583 |
| hsa-miR-30a-3p | 2489 | 10664 | 113 | 1680 | 2067 | 7962 | 145 | 1742 |
| hsa-miR-486-5p | 555 | 6576 | 904 | 197 | 579 | 11707 | 1506 | 251 |
| hsa-miR-582-3p | 1 | 251 | 5 | 159 | 3 | 155 | 5 | 139 |
| hsa-miR-196a-5p | 12053 | 16087 | 372 | 142 | 8892 | 8783 | 377 | 160 |
| hsa-miR-1271-5p | 4693 | 7648 | 31 | 100 | 4264 | 7417 | 32 | 143 |
| hsa-miR-379-5p | 27764 | 5420 | 12 | 79 | 24591 | 4251 | 30 | 77 |
| hsa-miR-409-5p | 11783 | 2226 | 8 | 69 | 9604 | 2230 | 13 | 69 |
| hsa-miR-411-5p | 17829 | 2815 | 3 | 43 | 17984 | 2428 | 16 | 36 |

### Example 5: Generation of miRNA-Regulated Plasmid Vectors for the Expression of GFP

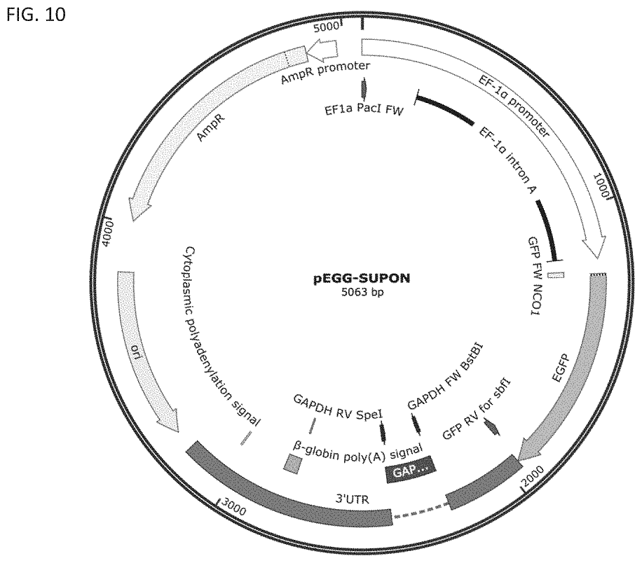

A pSUPON plasmid containing unmethylated GFP under the control of SV40 promoter (FIG. 2A) was used to construct an miRNA-regulated vector. Specifically, unmethylated GFP was replaced by eGFP for a stronger expression and the SV-40 promoter was replaced with a stronger EF1alpha promoter. Additionally, the 3′ UTR from the GAPDH housekeeping gene was cloned into the 3′ UTR of eGFP. All cloning reactions were verified via either PCR or sequencing. This vector was called pEGG-SUPON (EGG for EF1-GFP-GAPDH) (FIG. 10).

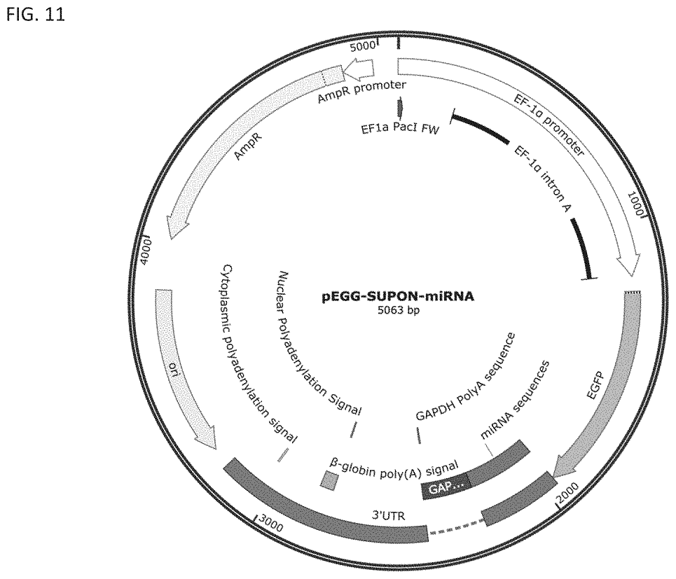

To make this vector regulated by miRNA sequences, four copies of an MBS specific for miR205-5p were inserted in the 3′ UTR of eGFP before the GAPDH sequence (FIG. 11). Similar vectors were generated, each vector containing four copies of an MBS specific for miR205-3p, miR200c-3p, miR224, miR577, or miR629. These six pEGG-SUPON-miRNA vectors were transfected into MCF7, BT549, and MCF10A depending on the respective cell line's miRNA profile and were observed for GFP expression (Data not shown).

### Example 6: Generation of miRNA-Regulated Plasmid Vectors to Induce Cell Death in Breast Cancer Cells

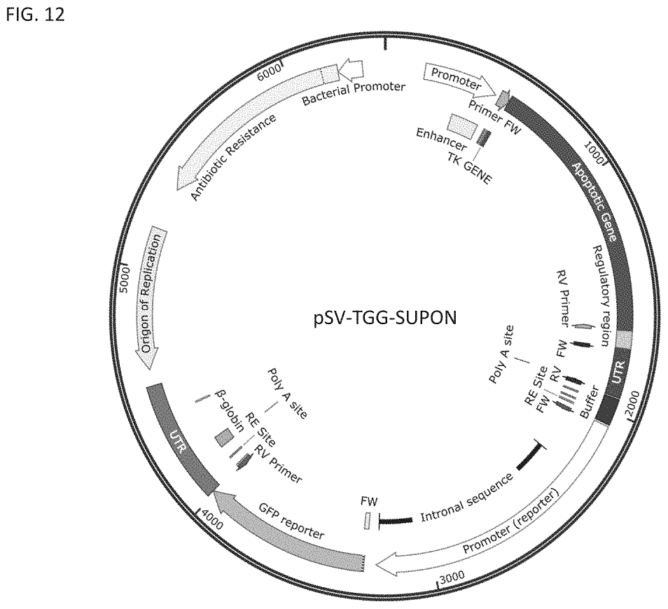

For this experiment, a pSUPON plasmid containing unmethylated GFP under the control of SV40 promoter (FIG. 2A) was modified as follows. HSVTK1 was cloned in place of GFP. GAPDH 3′ UTR was also cloned into the 3′ UTR of HSVTK. EF1alpha and eGFP were cloned downstream of the GAPDH 3′ UTR for identification of transfected cells. This vector is referred to as pSV-TGG-SUPON (TGG for TK-GAPDH-GFP) (FIG. 12). To make this vector regulated by miRNA sequences, four copies of an MBS specific for miR205-5p were inserted in the 3′ UTR of HSVTK1 before the GAPDH sequence. Five similar vectors were generated, each vector containing four copies of an MBS specific for miR205-3p, miR200c-3p, miR224, miR577, or miR629.

Additionally, a new vector with a stronger promoter amplifying HSVTKa was created. This new vector swapped out SV-40 for the CAG promoter, and was dubbed pCAG-TGG-SUPON.

A cell killing experiment using three of the six miRNA-regulated pSV-TGG-SUPON vectors was conducted. Briefly, the vectors were transfected into MCF10A cells (healthy breast cell line) and BT549 cells (triple negative breast cancer cell line).

The vector contains two translated regions: Herpes Thymadine Kinase (TK; regulated by miRNAs expressed in healthy cells) and GFP (not regulated by miRNAs). The TK gene of each transfected vector was targeted by four copies of a single miRNA not present in BT549 but present in MCF10A: miR205-3p, miR205-5p, or miR200C. TK was not regulated in the positive control vector. GFP was used as a reference for transfected cells as it was not regulated by miRNAs, and therefore served as a marker for the presence of the vector.

TK metabolizes a prodrug, gancyclovir, and the metabolite blocks DNA synthesis. The two components, TK and Gancyclovir, are not toxic on their own but only in combination. Thus, GFP+ cancer cells were expected to show cell death when treated with gancyclovir and GFP+ healthy cells were expected to survive when treated with gancyclovir.

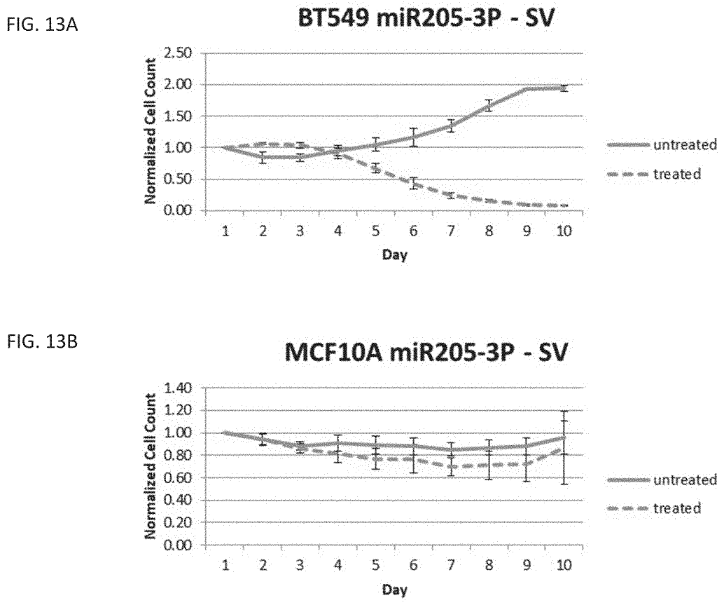

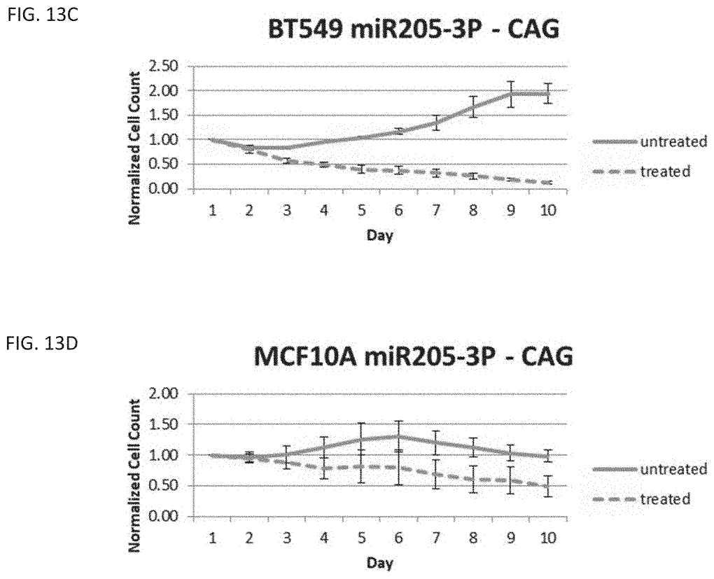

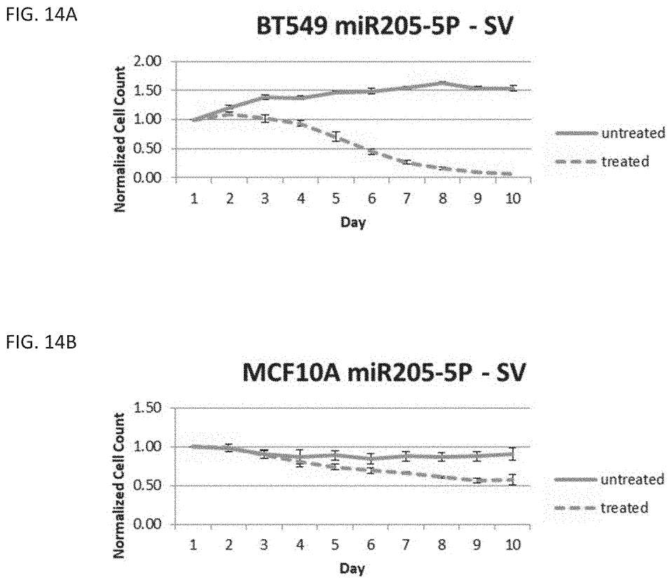

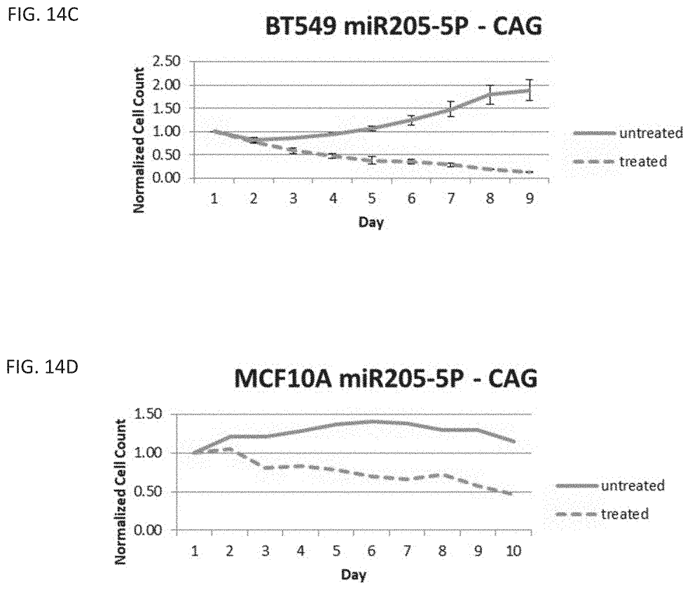

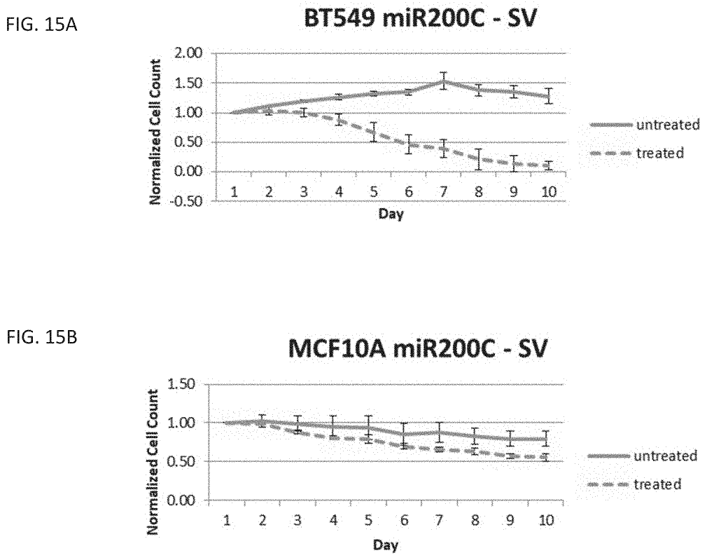

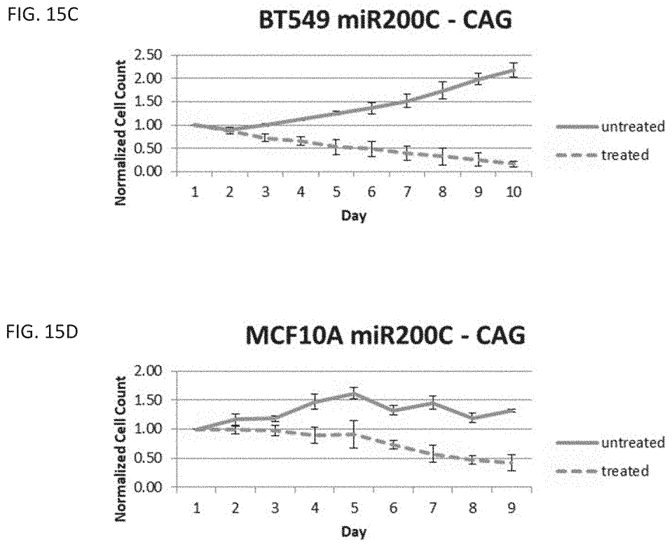

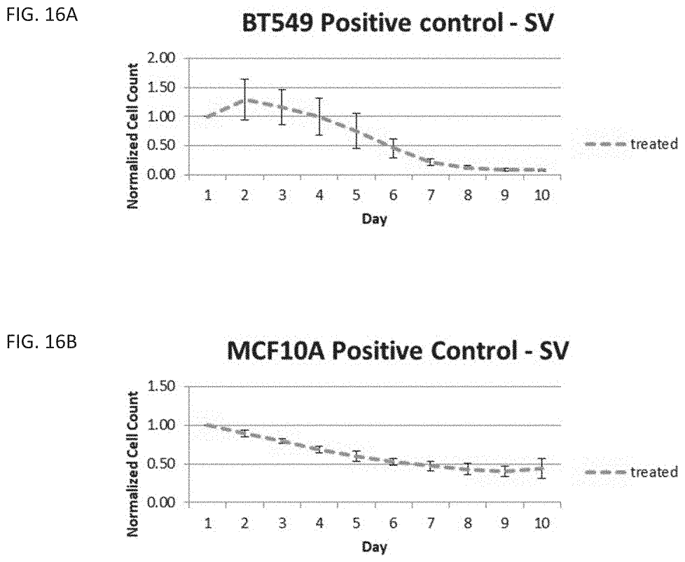

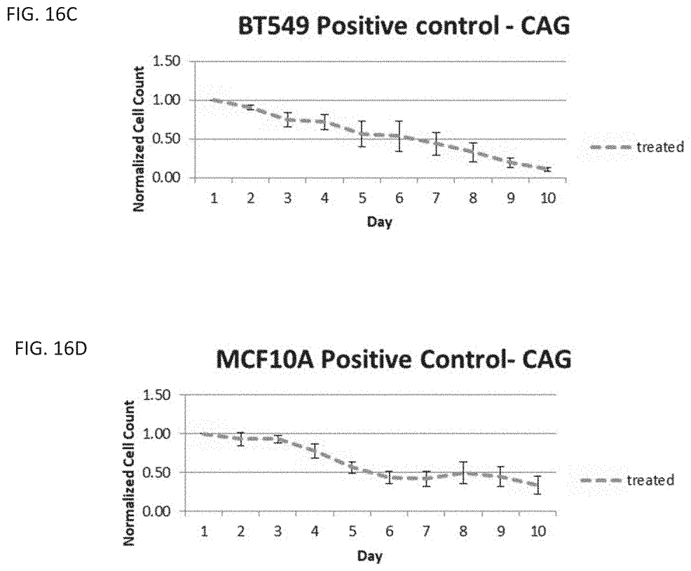

Following transfection, GFP+ cells were counted over the course of 10 days in untreated cells and cells treated with gancyclovir. FIGS. 13-15 show the total GFP cell count in respective cell lines transfected with the miRNA-regulated vectors normalized to day 1 in order to track expression over time. FIG. 16 show the total GFP cell count in respective cell lines transfected with the positive control vectors normalized to day 1.

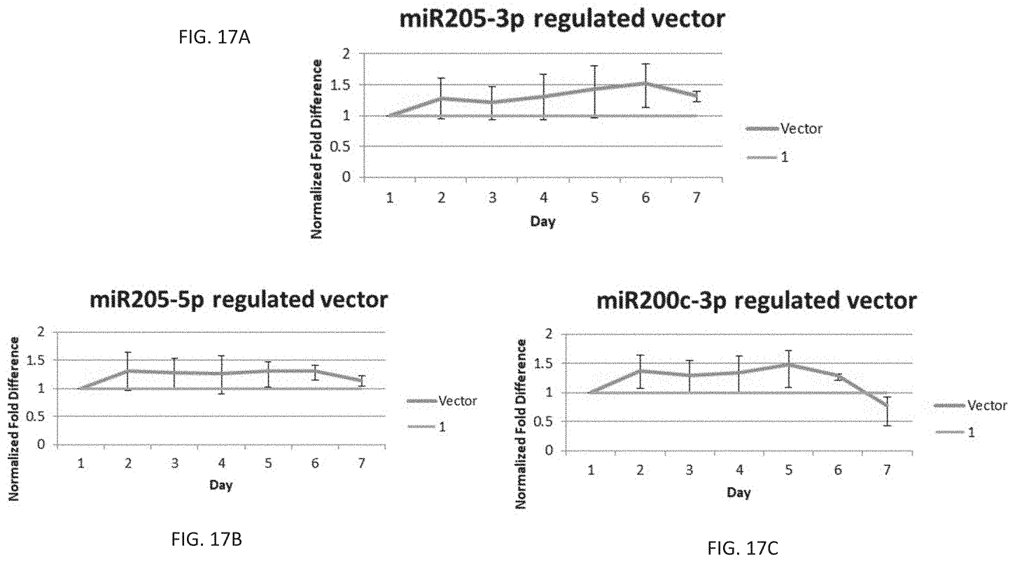

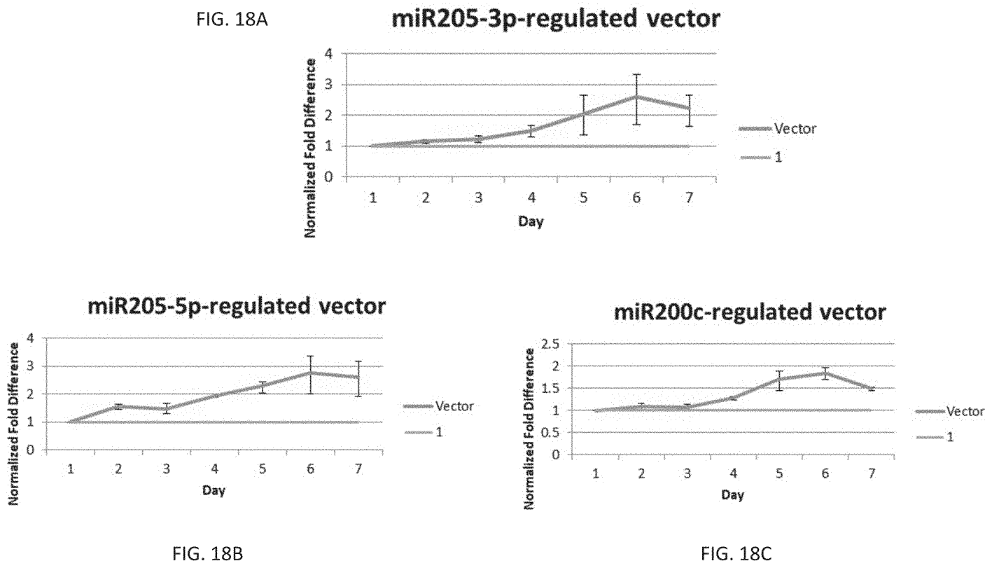

Normalized fold differences in cell killing between MCF10A and BT549 are shown in FIG. 17 (vectors with the SV40 promoter) and FIG. 18 (vectors with the CAG promoter). Values >1 indicate higher activity in cancer cells than healthy cells. The miRNA-regulated vectors with the SV40 promoter showed about 1-2 fold killing of cancer cells over the healthy cells (FIG. 17). The miRNA-regulated vectors with the CAG promoter showed about 2-3 fold killing of cancer cells over the healthy cells (FIG. 18).

1. A vector comprising:
(a) a first deoxyribonucleic acid (DNA) sequence comprising a transgene; and
(b) a second deoxyribonucleic (DNA) acid sequence comprising a microRNA binding domain (MBD); wherein the MBD comprises one or more microRNA binding sites (MBSs), wherein each MBS is specific for a microRNA (miR) that is present in a non-breast cancer cell and is not present or is downregulated in a breast cancer cell, and wherein the one or more MBSs are specific for one or more microRNAs selected from one of the combinations presented in Tables 1-4.

2. A vector comprising:
(a) a first deoxyribonucleic acid (DNA) sequence comprising a transgene; and
(b) a second deoxyribonucleic acid (DNA) sequence comprising a microRNA binding domain (MBD); wherein the MBD comprises one or more microRNA binding sites (MBSs), and wherein each MBS is specific for a microRNA (miR) that is present in a non-breast cancer cell and is not present or is downregulated in an early stage breast cancer cell or a late stage breast cancer cell.

3. (canceled)

4. The vector of claim 1, wherein the MBD comprises 1-12 MBSs.

5-7. (canceled)

8. The vector of claim 5, wherein the length of each MBS is about 6-33 nucleotides, about 6-30 nucleotides, about 6-27 nucleotides, about 6-25 nucleotides, about 6 to 23 nucleotides, about 6 to 20 nucleotides, about 6 to 18 nucleotides, about 6 to 15 nucleotides, about 6 to 13 nucleotides, about 6 to 11 nucleotides, or about 6 to 8 nucleotides.

9. The vector of claim 1, wherein the breast cancer cell is a late stage breast cancer cell, and wherein the one or more MBSs are specific for one or more microRNAs selected from the group consisting of: miR-629, miR-200C, miR-203A, miR-4760, miR-429, miR-95, miR-489, and combinations thereof, or selected from the group consisting of: miR-125a, miR-99b, miR-182, miR-93, miR-148b, miR-425, miR-30d, miR-26b, miR-484, miR-96, miR-185, miR-25, miR-203a, miR-454, miR-7, miR-23b, miR-342, miR-421, miR-106b, miR-141, miR-95, miR-345, miR-429, miR-542, miR-200b, miR-200a, miR-489, miR-618, miR-653, and combinations thereof, or selected from the group consisting of: miR-205, miR-200C, miR-510, and combinations thereof.

10-11. (canceled)

12. The vector of claim 1, wherein the breast cancer cell is an early stage breast cancer cell, and wherein the one or more MBSs are specific for one or more microRNAs selected from the group consisting of: miR-452, miR-224, miR-100, miR-31, miR-10A, and combinations thereof, or selected from the group consisting of: miR-224, miR-577, miR-452, miR-221, miR-100, miR-205, miR-31, and combinations thereof, or selected from the group consisting of: miR-221, miR-100, miR-22, miR-29a, miR-320a, miR-222, miR-31, miR-30c, miR-135b, miR-362, miR-146a, miR-221, miR-10a, miR-30a, miR-30a, miR-486, miR-582, miR-196a, miR-1271, miR-379, miR-409, miR-411, and combinations thereof.

13-21. (canceled)

22. The vector of claim 1, wherein the one or more MBSs are specific for one or more microRNAs selected from the group consisting of: miR-100, miR-138, miR-221, miR-222, miR-205, and combinations thereof, or selected from the group consisting of: miR-200, miR-205, miR-92a, miR-20a, miR-378a, miR-19b, miR-17, miR-183, miR-92b, miR-181b, miR-19a, miR-18a, miR-708, miR-92a-1, miR-584, miR-514a, miR-944, miR-205, and combinations thereof, or selected from the group consisting of: let-7b, miR-423, miR-423, miR-34c, miR-34a, and miR-296, miR-200c, miR-205, miR-92a, miR-20a, miR-378a, miR-19b, miR-17, miR-183, miR-92b, miR-181b, miR-19a, miR-18a, miR-708, miR-92a-1, miR-584, miR-514a, miR-944, and miR-205, miR-152, miR-455, miR-218, miR-143, miR-889, miR-138, miR-382, miR-199a, miR-487b, miR-134, miR-199a, miR-369, miR-494, miR-381, miR-10b, miR-145, miR-410, miR-199b, miR-329, miR-654, miR-376c, miR-409, miR-199b, miR-758, miR-369, miR-495, miR-145, miR-379, miR-323a, miR-377, miR-411, miR-487a, miR-539, miR-323b, miR-380, miR-412, miR-655, miR-1185-1, miR-127, miR-337, miR-382, miR-485, miR-654, miR-143, miR-370, miR-376a, miR-377, miR-432, miR-485, miR-543, miR-10b, miR-1185-2, miR-136, miR-136, miR-154, miR-154, miR-214, miR-214, miR-299, miR-299, miR-337, miR-431, miR-433, miR-490, miR-490, miR-493, miR-493, miR-539, miR-656, miR-665, and combinations thereof, or selected from the group consisting of: miR-629, miR-200C, miR-203A, miR-4760, miR-429, miR-95, miR-489, miR-205, miR-510, miR-34c, miR-203c, and combinations thereof, or selected from the group consisting of: miR-452, miR-224, miR-100, miR-31, miR-10A, miR-577, miR-221, miR-205, miR-34c, and combinations thereof.

23-27. (canceled)

28. The vector of claim 1, wherein the second deoxyribonucleic acid sequence is 3′ or 5′ of the first deoxyribonucleic acid sequence.

29. (canceled)

30. The vector of claim 28, wherein the second deoxyribonucleic acid sequence is 3′ of the first deoxyribonucleic acid sequence, and the vector comprising a third deoxyribonucleic acid sequence 3′ of the second deoxyribonucleic acid sequence, wherein the third deoxyribonucleic acid sequence comprises one or more 3′ untranslated regions (3′-UTRs) of one or more genes, wherein the 3′-UTR provides a translation efficiency of −0.3 to −0.0.8 where the translation efficiency is defined as the ratio of ribosome protected fragments (RPF) to the abundance of ribonucleic acids (RNA), wherein the one or more genes are one or more housekeeping or cytoskeleton genes selected from the group consisting of: GAPDH, α-tubulin, and β-tubulin, or the one or more genes are selected from the group consisting of: Rp132, HSP70a, and CrebA.

31-33. (canceled)

34. The vector of claim 30, comprising a fourth deoxyribonucleic acid sequence 5′ of the first deoxyribonucleic acid sequence, wherein the fourth deoxyribonucleic acid sequence comprises a promoter.

35. The vector of claim 34, comprising a fifth deoxyribonucleic acid sequence 5′ of the first deoxyribonucleic acid sequence, wherein the fifth deoxyribonucleic acid sequence comprises a repressor element that facilitates further inhibition of the expression of the transgene in non-breast cancer cells.

36-37. (canceled)

38. The vector of claim 34, wherein the first deoxyribonucleic acid sequence and the second deoxyribonucleic acid sequence are under the control of the same promoter.

39. The vector of claim 38, wherein the first deoxyribonucleic acid sequence, the second deoxyribonucleic acid sequence, and the third deoxyribonucleic acid sequence are under the control of an identical promoter, and wherein the promoter is specifically expressed in breast cells.

40. (canceled)

41. The vector of claim 1, wherein the transgene encodes a therapeutic protein that inhibits proliferation and/or metastasis of breast cancer cells, and wherein the therapeutic protein is an apoptosis inducing protein selected from thymidine kinase, a caspase, a granzyme, an exotoxin, or a proapoptotic member of the Bcl-2 family.

42-43. (canceled)

44. The vector of claim 1, further comprising a second transgene, and the vector further comprising a microRNA binding domain (MBD) operably linked to the second transgene; wherein the MBD comprises one or more microRNA binding sites (MBSs), wherein each MBS is specific for a microRNA that is present in a non-breast cancer cell and is not present or is downregulated in a breast cancer cell.

45. (canceled)

46. The vector of claim 1, wherein the transgene is a reporter transgene encoding a fluorescent protein or a luciferase.

47-52. (canceled)

53. A pharmaceutical composition comprising the vector of claim 1, and one or more pharmaceutically acceptable excipients.

54. A method for treating breast cancer in a subject in need thereof, comprising administering a therapeutically effective amount of the vector of claim 1, wherein the breast cancer is an early stage breast cancer or a late stage breast cancer.

55-56. (canceled)

57. A method for diagnosing breast cancer, comprising:
(a) introducing the vector of claim 46 into a breast tissue of a subject;
(b) measuring the expression of the reporter transgene;
(c) comparing the expression of the reporter transgene to a control; and
(d) diagnosing the subject as having breast cancer or not having breast cancer.

58-59. (canceled)

60. The method of claim 57, wherein the method comprises introducing the vector into a breast biopsy sample obtained from a subject.
